SEQUENCE LISTING (45 sequences)

SEQ ID NO: 1 - protein, 118 aa, Artificial Sequence
  Description of Artificial Sequence Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 2 - protein, 108 aa, Artificial Sequence
  Description of Artificial Sequence Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 3 - protein, 305 aa, Artificial Sequence
  Description of Artificial Sequence Synthetic polypeptide
METDTLLLWVLLLWVPGSTGDDIRRADIVMTQTTLSLPVSLGDQASISCRSSQSIVHSNGNTYLGWYLQKPGQSPKLLIYKVSNRFSGVPDRFSGTGSGTDFTLKISRVEAEDLGVYYCFQGSHVPYTFGGGTKLEIKRADAAPTVSGSGGGSGGGSGGGSEAKLQESGPVLVKPGASVKMSCKASGYTFTDYYMNLVKQSHGKSLEWIGVINPYNGDTSYNQKFKGKATLTVDKSSSTAYMELNSLTSEDSAVYYCARYYGSWFAYWGQGTLITVSTAKTTPPSVYPLAPRSSREQKLISEEDL

SEQ ID NO: 4 - protein, 120 aa, Artificial Sequence [sequence omitted]

SEQ ID NO: 5 - protein, 111 aa, Artificial Sequence [sequence omitted]

SEQ ID NO: 6 - protein, 113 aa, Artificial Sequence [sequence omitted]

SEQ ID NO: 7 - protein, 107 aa, Artificial Sequence [sequence omitted]

SEQ ID NO: 8 - protein, 117 aa, Artificial Sequence [sequence omitted]

SEQ ID NO: 9 - protein, 107 aa, Artificial Sequence [sequence omitted]

SEQ ID NO: 10 - protein, 117 aa, Artificial Sequence
  Description of Artificial Sequence Synthetic polypeptide
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 11 - protein, 113 aa, Artificial Sequence
  Description of Artificial Sequence Synthetic polypeptide
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 12 - protein, 116 aa, Artificial Sequence
  Description of Artificial Sequence Synthetic polypeptide
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSS

SEQ ID NO: 13 - protein, 107 aa, Artificial Sequence
  Description of Artificial Sequence Synthetic polypeptide
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIK

SEQ ID NO: 14 - protein, 120 aa, Artificial Sequence
  Description of Artificial Sequence Synthetic polypeptide
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSS

SEQ ID NO: 15 - protein, 107 aa, Artificial Sequence
  Description of Artificial Sequence Synthetic polypeptide
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIK

SEQ ID NO: 16 - protein, 21 aa, Artificial Sequence
  Description of Artificial Sequence Synthetic peptide
LCYLLDGILFIYGVILTALFL

SEQ ID NO: 17 - protein, 27 aa, Artificial Sequence
  Description of Artificial Sequence Synthetic peptide
FWVLVVVGGVLACYSLLVTVAFIIFWV

SEQ ID NO: 18 - protein, 28 aa, Artificial Sequence
  Description of Artificial Sequence Synthetic peptide
MFWVLVVVGGVLACYSLLVTVAFIIFWV

SEQ ID NO: 19 - protein, 22 aa, Artificial Sequence
  Description of Artificial Sequence Synthetic peptide
MALIVLGGVAGLLLFIGLGIFF

SEQ ID NO: 20 - protein, 21 aa, Artificial Sequence
  Description of Artificial Sequence Synthetic peptide
IYIWAPLAGTCGVLLLSLVIT

SEQ ID NO: 21 - protein, 23 aa, Artificial Sequence
  Description of Artificial Sequence Synthetic peptide
IYIWAPLAGTCGVLLLSLVITLY

SEQ ID NO: 22 - protein, 24 aa, Artificial Sequence
  Description of Artificial Sequence Synthetic peptide
IYIWAPLAGTCGVLLLSLVITLYC

SEQ ID NO: 23 - protein, 93 aa, Artificial Sequence
  Description of Artificial Sequence Synthetic polypeptide
SALSNSIMYFSHFVPVFLPAKPTTTPAPRPPTPAPTIASQPLSLRPEACRPAAGGAVHTRGLDFACDIYIWAPLAGTCGVLLLSLVITLYCNH

SEQ ID NO: 24 - protein, 86 aa, Artificial Sequence
  Description of Artificial Sequence Synthetic polypeptide
MYFSHFVPVFLPAKPTTTPAPRPPTPAPTIASQPLSLRPEACRPAAGGAVHTRGLDFACDIYIWAPLAGTCGVLLLSLVITLYCNH

SEQ ID NO: 25 - protein, 24 aa, Artificial Sequence
  Description of Artificial Sequence Synthetic peptide
IISFFLALTSTALLFLLFFLTLRF

SEQ ID NO: 26 - protein, 187 aa, Artificial Sequence
  Description of Artificial Sequence Synthetic polypeptide
KFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 27 - protein, 257 aa, Artificial Sequence
  Description of Artificial Sequence Synthetic polypeptide
EVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 28 - protein, 15 aa, Artificial Sequence
  Description of Artificial Sequence Synthetic peptide
GGGGSGGGGSGGGGS

SEQ ID NO: 29 - protein, 10 aa, Artificial Sequence
  Description of Artificial Sequence Synthetic peptide
GGGGSGGGGS

SEQ ID NO: 30 - protein, 5 aa, Artificial Sequence
  Description of Artificial Sequence Synthetic peptide
GGGGS

SEQ ID NO: 31 - protein, 10 aa, Artificial Sequence
  Description of Artificial Sequence Synthetic peptide
GGGSSGGGSG

SEQ ID NO: 32 - protein, 12 aa, Artificial Sequence
  Description of Artificial Sequence Synthetic peptide
ESKYGPPCPSCP

SEQ ID NO: 33 - protein, 12 aa, Artificial Sequence
  Description of Artificial Sequence Synthetic peptide
ESKYGPPCPPCP

SEQ ID NO: 34 - protein, 22 aa, Artificial Sequence
  Description of Artificial Sequence Synthetic peptide
ESKYGPPCPPCPGGGSSGGGSG

SEQ ID NO: 35 - protein, 39 aa, Artificial Sequence
  Description of Artificial Sequence Synthetic polypeptide
IEVMYPPPYLDNEKSNGTIIHVKGKHLCPSPLFPGPSKP

SEQ ID NO: 36 - protein, 48 aa, Artificial Sequence
  Description of Artificial Sequence Synthetic polypeptide
AKPTTTPAPRPPTPAPTIASQPLSLRPEACRPAAGGAVHTRGLDFACD

SEQ ID NO: 37 - protein, 45 aa, Artificial Sequence
  Description of Artificial Sequence Synthetic polypeptide
TTTPAPRPPTPAPTIASQPLSLRPEACRPAAGGAVHTRGLDFACD

SEQ ID NO: 38 - protein, 129 aa, Artificial Sequence
  Description of Artificial Sequence Synthetic polypeptide
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 107 aa, Artificial Sequence
  Description of Artificial Sequence Synthetic polypeptide
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 41 aa, Artificial Sequence
  Description of Artificial Sequence Synthetic polypeptide
RSKRSRLLHSDYMNMTPRRPGPTRKHQYPYAPPRDFAAYRS

SEQ ID NO: 41 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 42 - protein, 42 aa, Artificial Sequence
  Description of Artificial Sequence Synthetic polypeptide
ALYLLRRDQRLPPDAHKPPGGGSFRTPIQEEQADAHSTLAKI

SEQ ID NO: 43 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 1803 bp, Artificial Sequence
  Description of Artificial Sequence Synthetic polynucleotide
atggagacagacacactcctgctatgggtactgctgctctgggttccaggttccactggtgacgatatcaggcgcgccgacattgtgatgacccagactacactttccctgcctgtcagtcttggagatcaagcctccatctcttgcagatctagtcagagcattgtacatagtaatggaaacacctatttaggatggtacctgcagaaaccaggccagtctccaaagctcctgatctacaaagtttccaaccgattttctggggtcccagacaggttcagtggcactggatcagggacagatttcacactcaagatcagcagagtggaggctgaggatctgggagtttattactgctttcaaggttcacatgttccttacacgttcggaggggggaccaagctggaaataaaacgggctgatgctgcaccaactgtatccggatccggaggtgggagtggtggcggaagtggcggagggagcgaggcaaagctgcaggagtctggacctgtgctggtgaagcctggggcttcagtgaagatgtcctgtaaggcttctggatacacattcactgactactatatgaacttggtgaagcaaagccatggaaagagccttgagtggattggagttattaatccttataacggtgatactagctacaaccagaagttcaagggcaaggccacattgactgttgacaagtcctccagcacagcctacatggagctcaacagcctgacatctgaggactctgcagtctattactgtgcaagatactatggttcctggtttgcttactggggccaagggactctgatcactgtctctacagccaaaacaacacccccatcagtctatccactggcccctagatcttctcgagaacaaaaactcatctcagaagaggatctgggtggcggaggttctggtggcggaggttctggtggcggaggttcttcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctaa

SEQ ID NO: 45 - protein, 600 aa, Artificial Sequence
  Description of Artificial Sequence Synthetic polypeptide
METDTLLLWVLLLWVPGSTGDDIRRADIVMTQTTLSLPVSLGDQASISCRSSQSIVHSNGNTYLGWYLQKPGQSPKLLIYKVSNRFSGVPDRFSGTGSGTDFTLKISRVEAEDLGVYYCFQGSHVPYTFGGGTKLEIKRADAAPTVSGSGGGSGGGSGGGSEAKLQESGPVLVKPGASVKMSCKASGYTFTDYYMNLVKQSHGKSLEWIGVINPYNGDTSYNQKFKGKATLTVDKSSSTAYMELNSLTSEDSAVYYCARYYGSWFAYWGQGTLITVSTAKTTPPSVYPLAPRSSREQKLISEEDLGGGGSGGGGSGGGGSSALSNSIMYFSHFVPVFLPAKPTTTPAPRPPTPAPTIASQPLSLRPEACRPAAGGAVHTRGLDFACDIYIWAPLAGTCGVLLLSLVITLYCNHKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

(19) United States
(12) Patent Application Publication
(10) Pub. No.: US 2022/0162644 A1
(43) Pub. Date: May 26, 2022

(54) DELIVERY VECTORS AND PARTICLES FOR EXPRESSING CHIMERIC RECEPTORS AND METHODS OF USING THE SAME

(71) Applicant: ORBIS HEALTH SOLUTIONS, LLC, Greenville, SC (US)

(72) Inventor: Thomas E. Wagner, Greenville, SC (US)

(73) Assignee: ORBIS HEALTH SOLUTIONS, LLC, Greenville, SC (US)

(21) Appl. No.: 17/614,490

(22) PCT Filed: May 28, 2020
(86) PCT No.: PCT/US2020/034960

§ 371 Date: Nov. 26, 2021

Related U.S. Application Data
(60) Provisional application No. 62/854,082, filed on May 29, 2019.

(51) Int. Cl. C12N 15/86; C07K 14/705; A61K 48/00

(52) U.S. Cl. CPC C12N 15/86; C07K 14/70503; C12N 2740/16043; C07K 2319/01; C12N 2710/10343; A61K 48/005

(57) ABSTRACT

The present disclosure provides delivery vectors for expressing a chimeric receptor in a monocytic cell, such as a macrophage or dendritic cell. The chimeric receptor may specifically bind to a particular antigen or target molecule, such as an immune checkpoint protein or OX40. The disclosed delivery vectors can be used to treat cancer in a subject by expressing in vivo a chimeric receptor on the surface of the subject's monocytic cells.

### CROSS REFERENCE TO RELATED APPLICATION

This application claims priority under 35 U.S.C. § 119(e) to U.S. provisional application 62/854,082, filed May 29, 2019, the entire contents of which are incorporated herein by reference.

### FIELD OF INVENTION

The present disclosure relates generally to the field of cancer therapy, and, in particular, targeted monocyte-based cell therapy. The disclosure provides compositions and methods for effectively delivering a gene encoding a chimeric receptor to monocytic cells, such as macrophages, and other immune cells. The disclosure further provides methods of treating cancer by administering to a patient a particle comprising a vector that encodes a chimeric receptor, such that the chimeric receptor is expressed in vivo, thereby activating the patient's immune system to attach the cancer when the chimeric receptor binds its target molecule or antigen.

### BACKGROUND OF THE INVENTION

The following discussion is merely provided to aid the reader in understanding the disclosure and is not admitted to describe or constitute prior art thereto.

The immune system is made up of a variety of types of cells that are able to detect the presence of pathogens or pathologic cells in the body and remove them from the body. Sometimes this occurs by a foreign agent being enveloped by immune system cells and destroyed or carried out of the body. If living host cells have been invaded by a bacterial cell or virus, the immune system cells may target and destroy that infected cell.

For example, monocytic cells normally patrol the body in search of foreign, non-self-antigens, such as bacteria. Monocytic cells phagocytize bacteria, which are then digested to smaller antigenic portions in the lysosome. The resultant bacterial antigens are cycled back to the cell surface of these cells for presentation to the humoral and cellular arms of the immune system. Furthermore, monocytic cells can also detect, target, and destroy pathologic cells that have become damaged or genetically mutated. Cancer cells represent one example of such pathologic cells that can be killed by cells of the immune system.

Monocytes can differentiate into macrophages or dendritic cells after migrating from the blood stream into particular tissues. Importantly, many solid tumors have a vast presence of macrophage cells within the tumor bed. These tumor-associated macrophages (TAMs) are attracted to the hypoxic and/or necrotic microenvironments of the tumor, where they can serve to promote tumor growth and progression through various pathways, such as activation of nuclear factor-kappa B (NF-κB) and the release of pro-angiogenic signals (e.g., VEGF).

Moreover, in many situations, cancer cells are able to evade the patient's innate immune system. Under normal circumstance, T-cells will recognize particular antigens via a T-cell receptor (TCR), which is “primed” when the TCR on the surface of the T cell binds to a complex on the surface of the antigen-presenting cell, usually a dendritic cell, macrophage, or B cell, that contains the TCR's specific antigen. Next, the T cell must receive a co-stimulatory signal from the antigen-presenting cell. This is most commonly provided by engagement of the CD28 receptor on the T cell with either of its ligands, B7-1 and B7-2 (also called CD80 and CD86, respectively). When this process fails to occur, the innate immune system develops a tolerance to the cancer cells.

Thus, it would be beneficial to take advantage of TAMs in such a way that would reverse their innate pro-tumoral activity and instead function to stimulate the immune system to attack tumor cells through checkpoint inhibition and cytokine production.

### SUMMARY OF THE INVENTION

Described herein are compositions and methods for treating tumors using monocyte-specific bead vectors for directing expression of therapeutic proteins.

In one aspect, the disclosure provides delivery vectors comprising: (i) a base particle and (ii) a non-infectious virus attached to the outside of the particle, wherein the non-infectious virus comprises a nucleic acid encoding a chimeric receptor comprising a target binding domain, a transmembrane domain, and an intracellular signaling domain.

In some embodiments of the foregoing aspect, the nucleic acid encoding the chimeric receptor is comprised within an expression vector. In some embodiments, the expression vector comprises a T7 promoter or a hypoxia-induced promoter. In some embodiments, the expression vector comprises SEQ ID NO: 44.

In some embodiments of the foregoing aspect, the base particle is a yeast cell wall particle (YCWP). In some embodiments the YCWP is loaded with a biological material, such as a tumor lysate.

In some embodiments of the foregoing aspect, the base particle is a bead, such as a ferro-magnetic particle, a microbead, or a microsphere.

In some embodiments of the foregoing aspect, the delivery vector is a size that allows it to be preferentially phagocytized by a monocytic cell, such as a macrophage or, more specifically, a tumor-associated macrophage (TAM).

In another aspect, the disclosure provides monocytic cells comprising a chimeric receptor expressed on its surface, the chimeric receptor comprising a target binding domain, a transmembrane domain, and an intracellular domain. In some embodiments, the cell is a macrophage (e.g., a tumor associated macrophage) or a dendritic cell.

In some embodiments of the foregoing aspects, the target binding domain of the chimeric receptor comprises an scFv that binds to an immune checkpoint protein. For example, the checkpoint protein may be selected from the group consisting of CTLA-4, PD-1, PD-L1, LAG3, B7.1, B7-H3, B7-H4, TIM3, VISTA, CD137, OX40, CD40, CD27, CCR4, GITR, NKG2D, and KIR. In some embodiments, the checkpoint protein is CTLA-4. In some embodiments, the target binding domain comprises an scFv comprising SEQ ID NO: 3 or SEQ ID NO: 3 with the IgK leader sequence removed. In some embodiments, the target binding domain comprises a variable heavy chain sequence of SEQ ID NO: 1 and a variable light chain sequence of SEQ ID NO: 2. In some embodiments, the checkpoint protein is PD-1. In some embodiments, the target binding domain comprises a variable heavy chain sequence and a variable light chain sequence corresponding to the respective variable heavy and light chain sequences of pembrolizumab, nivolumab, cemiplimab, spartalizumab, camrelizumab, or sintilimab. In some embodiments, the checkpoint protein is PD-L1. In some embodiments, the target binding domain comprises a variable heavy chain sequence and a variable light chain sequence corresponding to the respective variable heavy and light chain sequences of durvalumab, atezolizumab or avelumab.

In some embodiments of the foregoing aspects, the target binding domain is specific for OX40 (i.e., it specifically recognizes or binds to OX40). For example, in some embodiments, the target binding domain comprises an scFv comprising a variable heavy chain sequence and a variable light chain sequence corresponding to the respective variable heavy and variable light chain sequences of scFv may comprise the CDRs and/or variable domain regions of 9B12 (NCT01644968), MOXR0916, PF-04518600, MEDI0562, MEDI6469, MEDI6383, PF-04518600, or BMS 986178. In some embodiments, the target binding domain comprises an extracellular domain of OX40L.

In some embodiments of the foregoing aspects, the transmembrane domain comprises at least the transmembrane portion of a toll-like receptor, CD28, CD4, CD8, 4-1BB, CD27, ICOS, OX40, HVEM, or CD30. In some embodiments, the transmembrane domain comprises any one of SEQ ID NOs: 16-25.

In some embodiments of the foregoing aspects, the intracellular signaling domain comprises an intracellular domain of a toll-like receptor (TLR), such as TLR4 or TLR9. In some embodiments, the intracellular signaling domain comprises SEQ ID NO: 26 or SEQ ID NO: 27.

In some embodiments of the foregoing aspects, the non-infectious virus is an adenovirus (e.g., a recombinant adenovirus), lentivirus, or adeno-associated virus. In some embodiments, the non-infectious virus is also non-replicative.

In another aspect, the disclosure provides methods of treating cancer in a patient comprising administering to a patient with cancer the delivery vector of any one of foregoing embodiments. In another aspect, the disclosure provides methods of stimulating the immune system in a patient comprising administering to a patient with cancer the delivery vector of any of the foregoing embodiments.

In some embodiments of the disclosed methods, the delivery vector is administered intradermally. In some embodiments of the disclosed methods, the delivery vector is administered proximate to a target lymph node.

In some embodiments of the disclosed methods, the cancer comprises at least one tumor comprising a hypoxic microenvironment. In some embodiments, the at least one tumor comprises tumor-associated macrophages (TAMs). In some embodiments, the cancer comprises at least one solid tumor.

In some embodiments of the disclosed methods, the delivery vector is phagocytosed by a macrophage and the macrophage subsequently expresses the chimeric receptor on its surface.

The disclosure also provides delivery vectors according to any one of the foregoing embodiments for use as an anti-cancer agent.

The disclosure also provides delivery vectors according to any one of the foregoing embodiments for use in treating cancer in a subject comprising administering the delivery vector to the subject.

The disclosure also provides delivery vectors according to any one of the foregoing embodiments for use in stimulating the immune system in a subject comprising administering the delivery vector to the subject.

The disclosure also provides uses of any of the foregoing embodiments of the delivery vectors as anti-cancer agents.

The disclosure also provides uses of any of the foregoing embodiments of the delivery vectors for treating cancer in a subject comprising administering the delivery vector to the subject.

The disclosure also provides uses of any of the foregoing embodiments of the delivery vectors for stimulating the immune system in a subject comprising administering the delivery vector to the subject.

The foregoing general description and following detailed description are exemplary and explanatory and not limiting of the disclosure.

### BRIEF DESCRIPTION OF THE DRAWINGS

FIG. 1 shows the coding sequence and components of an exemplary chimeric receptor comprising an anti-CTLA-4 scFv, a Gly4/Ser1 linker, a human CD8 transmembrane domain, and a human TLR4 intracellular signaling domain.

FIG. 2 shows the amino acid sequence and components of an exemplary chimeric receptor comprising an anti-CTLA-4 scFv, a Gly4/Ser1 linker, a human CD8 transmembrane domain, and a human TLR4 intracellular signaling domain.

FIG. 3 shows an exemplary expression vector comprising a chimeric receptor fusion gene.

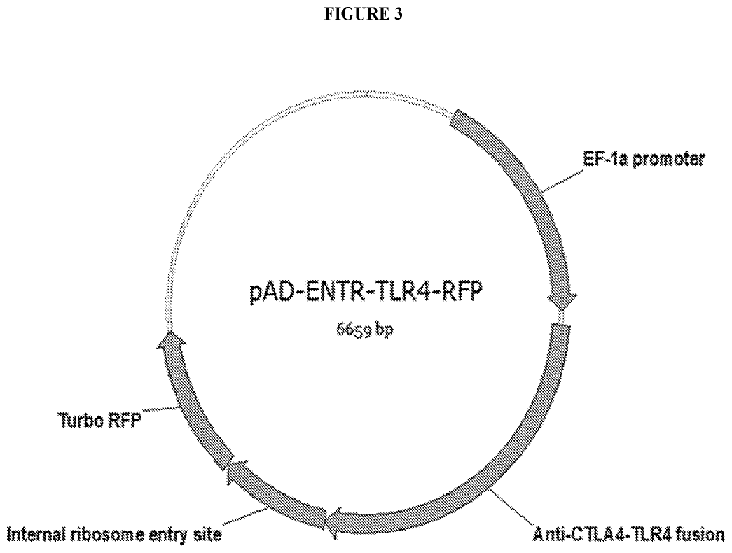

FIG. 4 shows an exemplary lentiviral vector that can be used in preparing the disclosed particles.

FIG. 5 shows the results of an IL-12-specific ELISA assay. In this assay, THP-1 cells expressing a CTLA4-specific chimeric receptor were exposed to various concentrations of recombinant CTLA4. The cells responded to CTLA4 exposure by expressing IL-12 in a concentration-dependent manner. IL-12 expression was not stimulated by exposure to LPS, which served as a positive control for TLR activation. P1 and P2 were positive controls of IL-12 and EB is a control provided by the ELISA Kit.

### DETAILED DESCRIPTION

In general, the present disclosure provides novel, targeted gene delivery vectors and methods of using the same to express an exogenous protein and treat cancer. In particular, the disclosure provides bead- or yeast cell wall particle (YCWP)-based delivery vectors for expressing a chimeric receptor (i.e., a chimeric antigen receptor (CAR) or modified toll-like receptor (TLR)) in monocytic cells, such as macrophages and dendritic cells. In particular, the disclosed compositions and methods can be used to express a chimeric receptor in tumor-associated macrophage (TAM) cells in the microenvironment of a tumor or a tumor-draining lymph node. The monocytic cells (e.g., TAMs) that express the disclosed chimeric receptors can stimulate the immune system to attack a tumor and overcome the immune tolerance that allows many tumors to avoid detection by the immune system.

More specifically, the disclosed chimeric receptors generally comprise an intracellular signaling domain derived from a toll-like receptor (TLR). TLRs are a class of single, membrane-spanning, non-catalytic receptors that play a key role in the innate immune system and are usually expressed on monocytic cells such as macrophages and dendritic cells. Under normal circumstances, TLRs recognize structurally conserved molecules derived from microbes and other foreign, non-self antigens, and upon recognizing such an antigen, TLRs can activate immune cell responses, including but not limited to the expression of cytokines. More specifically, TLRs recruit adapter proteins (proteins that mediate other protein-protein interactions) within the cytosol of the immune cell in order to propagate the antigen-induced signal transduction pathway. These recruited proteins are then responsible for the subsequent activation of other downstream proteins, including protein kinases (IKKi, IRAK1, IRAK4, and TBK1) that further amplify the signal and ultimately lead to the upregulation or suppression of genes that orchestrate inflammatory responses and other transcriptional events. Some of these events lead to cytokine production, proliferation, and survival, while others lead to greater adaptive immunity.

The disclosed chimeric receptors function as a modified TLR, by replacing the normal binding/antigen-recognition domain of a TLR and replacing it with an target binding domain that recognizes a checkpoint protein or another receptor or molecule involved in immune signaling (e.g., CTLA4, PD-1, PD-L1, OX40). Thus, the binding of such a chimeric receptor to its target molecule will result in a TLR activation to produce an aggressive anti-tumor immune response. While the response may vary depending on the intracellular TLR domain utilized for a given chimeric receptor, it is desirable to use the signaling domain of a TLR (e.g., TLR-4, TLR-9, etc.) that results in the expression of M1-type (i.e., pro-inflammatory) cytokines, such as IL-12, IFN-α, IRN-γ, TNF-α, IL-6, and/or IL-1β. As a result, expression and activation of the disclosed chimeric receptors by monocytic cells, such as macrophages, allows the chimeric receptor-expressing cells to overcome the immune suppressive environment fostered by most tumors without directly attacking or phagocytosing the tumor cells like a convention CAR T-cell. Moreover, because the putative mechanism of action of such receptors in indirect and based on propagating immune stimulation, it is not necessary for the chimeric receptor-expressing cells to physically contact the tumor or reside in the tumor microenvironment (although such localization is perfectly acceptable and will still function to destroy the target tumor). In fact, localization of the chimeric receptor-expressing cells in a tumor adjacent lymph node (such as a tumor-draining lymph node) is sufficient to destroy a tumor by producing an environment or milieu that is immune active.

Throughout this disclosure, various publications, patents and published patent specifications are referenced by an identifying citation. The disclosures of these publications, patents and published patent specifications are hereby incorporated by reference into the present disclosure to more fully describe the state of the art to which this disclosure pertains.

### Definitions

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term as well as the specified term. For example, “about 10” should be understood as meaning “10” as well as “9 to 11.”

As used herein, the term “antigen binding domain” may be used interchangeably with “target binding domain.” These terms should be understood as referring to the target molecule intended to be bound by the disclosed chimeric receptors (e.g., PD-1, PD-L1, CTLA4, OX-40, etc.). The terms should not be understood as implying that the target molecule is necessarily immunogenic or antigenic, per se, but merely that the disclosed receptor, which may comprise an antibody or antibody fragment as part of the binding domain, can bind to the target molecule in the same sense that an isolated antibody can bind its target antigen.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the phrases “therapeutically effective amount” means that a dose of the disclosed particles provides the specific pharmacological effect for which the drug is administered in a subject in need of such treatment, i.e. to reduce, ameliorate, or eliminate cancer/tumor growth, progression, or recurrence by activating the immune system. It is emphasized that a therapeutically effective amount of a particle will not always be effective in treating the cancer/tumors of every individual subject, even though such dosage is deemed to be a therapeutically effective amount by those of skill in the art. Those skilled in the art can adjust what is deemed to be a therapeutically effective amount in accordance with standard practices as needed to treat a specific subject and/or specific type of cancer or tumor. The therapeutically effective amount may vary based on the route of administration, site of administration, dosage form, the age and weight of the subject, and/or the subject's condition, including the progression, stage, and/or class of cancer or tumor at the time of treatment.

The terms “treatment” or “treating” as used herein with reference to cancer or tumors refer to reducing, ameliorating or eliminating cancer/tumor growth and/or progression, or causing caner/tumor cell death.

The terms “prevent” or “preventing” as used herein refer to stopping the formation of cancer/tumor cells or inhibiting the recurrence of cancer/tumor growth.

The terms “individual,” “subject,” and “patient” are used interchangeably herein, and refer to any individual mammalian subject, e.g., bovine, canine, feline, equine, or human.

The compositions and methods of the disclosure may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

### Chimeric Receptors

The disclosed delivery vectors are designed to deliver a nucleic acid encoding a chimeric receptor into target cell, such as a monocytic cell. For the purposes of the present disclosure, the chimeric receptors of the present disclosure comprise at least one target binding domain, a transmembrane domain, and an intracellular signaling domain. In some embodiments, a chimeric receptor may further comprise a hinge/linker domain and/or a co-stimulatory domain.

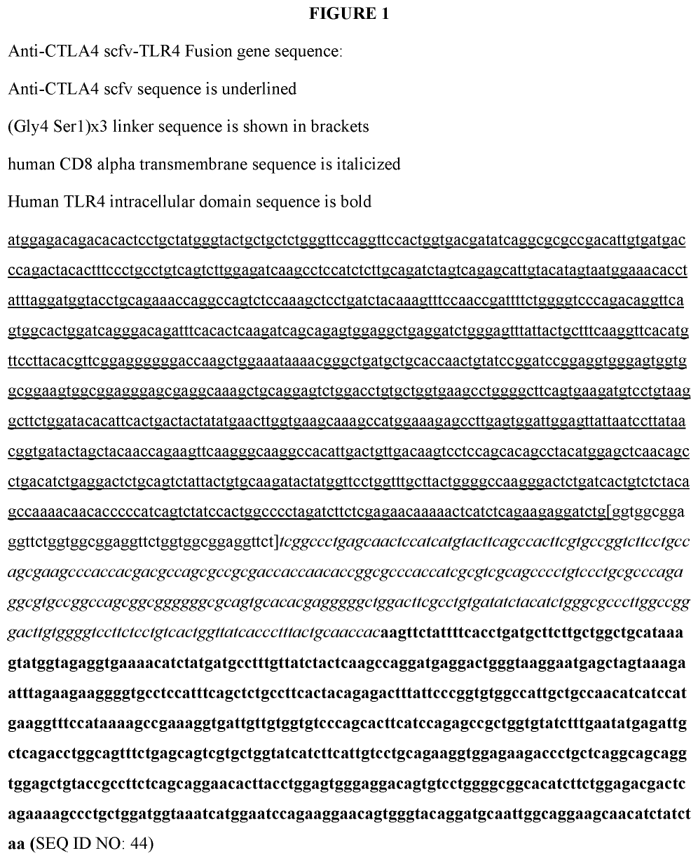

In some embodiments, the target binding domain may be an exogenous or non-natural sequence (e.g., an scFv fragment), while the remainder of the chimeric receptor sequence comprises a toll-like receptor (TLR) sequence. For example, an exemplary chimeric receptor may comprise an anti-CTLA-4 scFv connected to a CD8 transmembrane domain via a Gly4/Ser1 linker and a human TLR4 intracellular signaling domain. The coding sequence for such an exemplary chimeric receptor is shown in FIG. 1. In some embodiments, the chimeric receptor may comprise a transmembrane domain and/or an intracellular domain that were not derived from a TLR (e.g., a CDA signaling domain).

A. Target Binding Domain

The target binding domain of the disclosed chimeric receptors dictates the specificity of the receptor. In general, the target binding domain will comprise the variable domains of an antibody (e.g., an scFv domain), but in some embodiments, the target binding domain may comprise a peptide that binds to a targeted receptor, such as an extracellular domain of PD-1, an extracellular domain of CTLA-4, or an extracellular domain of OX40 or OX40L (also known as gp34, CD252, and TNFSF4). The PD-1 extracellular domain may be derived from human (NP_005009, NM_005018), mouse (NP_032824, NM_008798), bovine (NP_001277851, NM_001290922), or other animal origin. One of skill in the art will be able to identify a suitable extracellular domain of PD-1. For instance, human PD-1 is 288 amino acids in length, and amino acids 14-130 represent the extracellular domain, whereas murine PD-1 is also 288 amino acids but amino acids 21-169 represent the extracellular domain. The OX40 extracellular domain may be derived from human (NP_003318, NM_003327), mouse (NP_035789, NM_011659), or other animal origin. One of skill in the art will be able to identify a suitable extracellular domain of OX40. For instance, amino acids 1-191 of the N-terminus of human OX40 make up the extracellular domain. The OX40L extracellular domain may be derived from human (NP_003317, NM_003326; NM_001297562, NP_001284491) or other animal origin. One of skill in the art will be able to identify a suitable extracellular domain of OX40L. For instance, amino acids 1-133 of the N-terminus of human OX40L make ups the extracellular domain. An extracellular domain, such as the ligand binding domain of the receptor, of any of the inhibitory or co-stimulatory receptors listed in Table 1 below may also be incorporated into the disclosed chimeric receptors as a suitable target binding domain.

While it should be understood that a target binding domain with specificity for virtually any tumor-related antigen or immune pathway signaling molecule could be incorporated into the disclosed chimeric receptors, the preferred targets are immune checkpoint proteins or OX40.

Immune checkpoints are proteins involved in inhibitory pathways of the immune system, which, under normal conditions are crucial for maintaining self-tolerance and modulating the duration and amplitude of physiological immune responses in peripheral tissues in order to minimize collateral tissue damage in response to pathogenic infection. However, the expression of immune checkpoint proteins is often dysregulated by tumors as an important mechanism of immune resistance and immune evasion.

Because many of the immune checkpoints are initiated by ligand-receptor interactions, they can be readily blocked by antibodies or binding fragments specific for the checkpoint ligands and/or receptors. Thus, the target binding domain of the disclosed chimeric receptor may be designed to be specific for checkpoint proteins including, but not limited to, those proteins shown in Table 1, and the binding of the chimeric receptor to its target checkpoint protein will inhibit checkpoint signaling.

TABLE 1

| Target | Biological Function |
|---|---|
| CTLA-4 | Inhibitory Receptor |
| PD-1 | Inhibitory Receptor |
| PD-L1 | Ligand for PD1 |
| LAG3 | Inhibitory Receptor |
| B7.1 | Co-stimulatory Molecule |
| B7-H3 | Inhibitory Ligand |
| B7-H4 | Inhibitory Ligand |
| TIM3 | Inhibitory Receptor |
| VISTA | Inhibitory Receptor |
| CD137 | Co-stimulatory Molecule |
| OX40 | Co-stimulatory Receptor |
| CD40 | Co-stimulatory Molecule |
| CD27 | Co-stimulatory Receptor |
| CCR4 | Co-stimulatory Receptor |
| GITR | Co-stimulatory Receptor |
| NKG2D | Activating Receptor |
| KIR | Co-stimulatory Receptor |

CTLA4, cytotoxic T-lymphocyte-associated antigen 4; LAG3, lymphocyte activation gene 3; PD1, programmed cell death protein 1; PDL, PD1 ligand; TIM3, T cell membrane protein 3; VISTA, V-domain immunoglobulin (Ig)-containing suppressor of T-cell activation; KIR, killer IgG-like receptor.

For the purposes of this disclosure, the target binding domains that target the checkpoint proteins are not particularly limited. For instance, the target binding domain may comprise all or a portion of a human, chimeric, humanized, or non-human (e.g., mouse, rat, rabbit, sheep, goat, bovine, porcine, etc.) antibody. The parent antibodies (i.e., the antibody sequence used for incorporation into the chimeric receptor) may be IgG1, IgG2, IgG3, IgG4, IgA1, IgA2, IgE, or IgM, or variants or fragments thereof. In some embodiments, the target binding domain comprises a single chain Fv (scFv) antibody fragment (see e.g., Bird et al., Science, 242:423-26 (1988); Huston et al., Proc. Natl. Acad. Sci. USA, 85:5879-83 (1988)), particularly an scFv that binds to an immune checkpoint protein, including but not limited to, any of the immune checkpoint proteins recited in Table 1 above.

For example, in some embodiments, the chimeric receptor may comprise an anti-CTLA-4 scFv as its binding domain. An anti-CTLA-4 scFv may comprise the complementarity determining regions (CDRs) and/or variable domain regions of ipilimumab, which are shown in the table below.

Ipilimumab Sequences

| | | |
|---|---|---|
| Heavy | QVQLVESGGGVVQPGRSLRLSCAAS | SEQ |
| Chain | [symbol] MHWVRQAPGKGLEWVT | ID |
| Variable | F [symbol] [symbol] YYADSVKGRFTISRD | NO: |
| Region | NSKNTLYLQMNSLRAEDTAIYYCA | 1 |
| | [symbol] [symbol] WGQGTLVTVSS | |
| Light | EIVLTQSPGTLSLSPGERATLSCRA | SEQ |
| Chain | S [symbol] AWYQQKPGQAPRLLIY | ID |
| Variable | [symbol] SRATGIPDRFSGSGSGTDFTL | NO: |
| Region | TISRLEPEDFAVYYC | 2 |
| | [symbol] FGQGTKVEIK | |

*CDR sequences are shown in bold italics.

Further exemplary anti-CTLA4 scFv include, but are not limited to a scFv derived from the variable chain sequences of tremelimumab or a scFV comprising the sequence: [symbol] DIVMTQTTLSLPVSLGDQASISCRSSQSIVH SNGNTYLGWYLQKPGQSPKLLIYKVSNRFSGVPDRFSGTGSGTDFTLKISRVEAEDL GVYYCFQGSHVPYTFGGGTKLEIKRADAAPTVSGSGGGSGGGSGGGSEAKLQESG PVLVKPGASVKMSCKASGYTFTDYYMNLVKQSHGKSLEWIGVINPYNGDTSYNQK FKGKATLTVDKSSSTAYMELNSLTSEDSAVYYCARYYGSWFAYWGQGTLITVSTA KTTPPSVYPLAPRSSREQKLISEEDL (SEQ ID NO: 3; bold/italicized text represents an IgK leader sequence) The full length sequence of the antibody from which this scFv was derived from, as well as a nucleic acid sequence encoding the antibody are disclosed in US 2011/0044953, which is hereby incorporated by reference.

In some embodiments, the chimeric receptor may comprise an anti-PD-1 scFv as its binding domain. An anti-PD-1 scFv may comprise the CDRs and/or variable domain regions of pembrolizumab, nivolumab, cemiplimab, spartalizumab, camrelizumab, or sintilimab, which are shown in the table below.

Pembrolizumab Sequences

| | | |
|---|---|---|
| Heavy | QVQLVQSGVEVKKPGASVKVSCKASGYTFTN [symbol] WVRQAPGQGLEWMG | SEQ |
| Chain | [symbol] RVTLTTDSSTTTAYMELKSLQFDDTAVYYCAR [symbol] | ID NO: |
| Variable | [symbol] WGQGTTVTVSS | 4 |
| Region | | |
| Light | EIVLTQSPATLSLSPGERATLSC [symbol] WYQQKPGQAPRLLIYL | SEQ |
| Chain | [symbol] GVPARFSGSGSGTDFTLTISSLEPEDFAVYYC [symbol] FGGGTKV | ID NO: |
| Variable | EIK | 5 |
| Region | | |

Nivolumab Sequences

| | | |
|---|---|---|
| Heavy | QVQLVESGGGVVQPGRSLRLDCKASGITFSNSGMHWVRQAPGKGLEW [symbol] | SEQ |
| Chain | [symbol] RFTISRDNSKNTLFLQMNSLRAEDTAVYYCAT | ID NO: |
| Variable | [symbol] WGQGTLVTVSS | 6 |
| Region | | |
| Light | EIVLTQSPATLSLSPGERATLSC [symbol] WYQQKPGQAPRLLI [symbol] | SEQ |
| Chain | [symbol] TGIPARFSGSGSGTDFTLTISSLEPEDFAVYYC [symbol] FGQGTKVEIK | ID NO: |
| Variable | 7 | |
| Region | | |

Cemiplimab Sequences

| | | |
|---|---|---|
| Heavy | EVQLLESGGVLVQPGGSLRLSCAASGFTFSN [symbol] WVRQAPGKGLEWVS [symbol] | SEQ |
| Chain | [symbol] RFTISRDNSKNTLYLQMNSLKGEDTAVYYCVK [symbol] | ID NO: |
| Variable | [symbol] WGQGTLVTVSS | 8 |
| Region | | |
| Light | DIQMTQSPSSLSASVGDSITITC [symbol] NWYQQKPGKAPNLLIY [symbol] | SEQ |
| Chain | [symbol] GGVPSRFSGSGSGTDFTLTIRTLQPEDFATYYC [symbol] FGPGTVVDFR | ID NO: |
| Variable | 9 | |
| Region | | |

Spartalizumab Sequences

| | | |
|---|---|---|
| Heavy | EVQLVQSGAEVKKPGESLRISCKGSGYTFT [symbol] WVRQATGQGLEWMG [symbol] | SEQ |
| Chain | [symbol] RVTITADKSTSTAYMELSSLRSEDTAVYYCTR [symbol] | ID NO: |
| Variable | [symbol] WGQGTTVTVSS | 10 |
| Region | | |
| Light | EIVLTQSPATLSLSPGERATLSC [symbol] NFLTWYQQKPGQAPRLLI | SEQ |
| Chain | Y [symbol] GVPSRFSGSGSGTDFTFTISSLEAEDAATYYC [symbol] FGQG | ID NO: |
| Variable | TKVEIK | 11 |
| Region | | |

Camrelizumab Sequences

| | | |
|---|---|---|
| Heavy | EVQLVESGGGLVQPGGSLRLSCAASGFTFS [symbol] WVRQAPGKGLEWVA [symbol] | SEQ |
| Chain | [symbol] RFTISRDNAKNSLYLQMNSLRAEDTAVYYCAR [symbol] | ID NO: |
| Variable | [symbol] WGQGTTVTVSS | 12 |
| Region | | |
| Light | DIQMTQSPSSLSASVGDRVTITC [symbol] WYQQKPGKAPKLLIY [symbol] | SEQ |
| Chain | [symbol] DGVPSRFSGSGSGTDFTLTISSLQPEDFATYYC [symbol] FGGGTKVEIK | ID NO: |
| Variable | | 13 |
| Region | | |

Sintilimab Sequences

| | | |
|---|---|---|
| Heavy | QVQLVQSGAEVKKPGSSVKVSCKASGGTFS [symbol] WVRQAPGQGLEWMG [symbol] | SEQ |
| Chain | [symbol] RVAITVDESTSTAYMELSSLRSEDTAVYYCAR [symbol] | ID NO: |
| Variable | [symbol] WGQGTLVTVSS | 14 |
| Region | | |
| Light | DIQMTQSPSSVSASVGDRVTITC [symbol] WYQQKPGKAPKLLIS [symbol] | SEQ |
| Chain | [symbol] SGVPSRFSGSGSGTDFTLTISSLQPEDFATYYC [symbol] FGGGTKVEIK | ID NO: |
| Variable | | 15 |
| Region | | |

*CDR sequences are shown in bold italics.

In some embodiments, the chimeric receptor may comprise an anti-PD-L1 scFv as its binding domain. An anti-PD-L1 scFv may comprise the CDRs and/or variable domain regions of durvalumab, atezolizumab and avelumab.

In some embodiments, the chimeric receptor may comprise an anti-OX40 scFv as its binding domain. An anti-OX40 scFv may comprise the CDRs and/or variable domain regions of 9B12 (NCT01644968), MOXR0916, PF-04518600, MEDI0562, MEDI6469, MEDI6383, PF-04518600, or BMS 986178. OX40 is particularly desirable among the target molecules disclosed herein due to its unique signaling properties. For example, OX40 may expressed on multiple different types of T cells, and its function will vary depending on the cell type. When OX40 that is expressed on T effector or T helper cells binds its ligand, the cells are activated. But when OX40 that is expressed on T reg cells binds its ligand, the cells are inactivated. Accordingly, agonizing OX40 signaling would help to overcome the immune suppressive environments fosters by many tumors and creating an active immune environment by propagating an aggressive immune response.

B. Transmembrane Domain

The disclosed chimer receptors comprise a transmembrane domain connecting the target binding domain to the intracellular signaling domain. In general, human protein sequences are preferred for the purposes of a transmembrane domain of the present chimeric receptors. Various transmembrane domains that are commonly utilized in known chimeric antigen receptors (CARs) may be used here. For example, in some embodiments, the transmembrane domain may comprise at least the transmembrane portion of a toll-like receptor, CD28, CD4, CD8, 4-1BB, CD27, ICOS, OX40, HVEM, or CD30. Specific exemplary transmembrane domains are included in the table below, but are not intended to be limiting.

| SEQ | | |
|---|---|---|
| ID | | |
| NO: | Description | Sequence |
| 16 | CD3z | LCYLLDGILFIYGVILTALFL |
| 17 | CD28-1 | FWVLVVVGGVLACYSLLVTVAFI |
| | | IFWV |
| 18 | CD28-2 | MFWVLVVVGGVLACYSLLVTVA |
| | | FIIFWV |
| 19 | CD4 | MALIVLGGVAGLLLFIGLGIFF |
| 20 | CD8-1 | IYIWAPLAGTCGVLLLSLVIT |
| 21 | CD8-2 | IYIWAPLAGTCGVLLLSLVITLY |
| 22 | CD8-3 | IYIWAPLAGTCGVLLLSLVITLYC |
| 23 | CD8-4 | SALSNSIMYFSHFVPVFLPAKPTTT |
| | | PAPRPPTPAPTIASQPLSLRPEAC |
| | | RPAAGGAVHTRGLDFACDIYIWAP |
| | | LAGTCGVLLLSLVITLYCNH |
| 24 | CD8-5 | MYFSHFVPVFLPAKPTTTPAPRPP |
| | | TPAPTIASQPLSLRPEACRPAAGG |
| | | AVHTRGLDFACDIYIWAPLAGTC |
| | | GVLLLSLVITLYCNH |
| 25 | 4-1BB | IISFFLALTSTALLFLLFFLTLRF |

C. Intracellular Signaling Domain

The intracellular signaling domain of the chimeric receptor dictates the cellular response that the chimeric receptor produces. In general, human protein sequences are preferred for the purposes of an intracellular signaling domain of the present chimeric receptors. For example, in some embodiments, the chimeric receptor may comprise the intracellular signaling domain of a TLR, including TLR1, TLR2, TLR3, TLR4, TLR5, TLR6, TLR7, TLR8, TLR9, TLR10, TLR11, TLR12, and TLR13. Switching between the various TLRs will alter the cytokine response of the monocytic cell. The signaling domains of TLR4 and TLR9 are particularly useful for treating cancer, but for the purposed of the present disclosure, the chimeric receptor could alternatively comprise the signaling domain of TLR1, TLR2, TLR3, TLR5, TLR6, TLR7, TLR8, TLR10, TLR11, TLR12, or TLR13.

| SEQ | | |
|---|---|---|
| ID | | |
| NO: | Description | Sequence |
| 26 | TLR4 | KFYFHLMLLAGCIKYGRGENIYDAFVIYSS |
| | | QDEDWVRNELVKNLEEGVPPFQLCLHYRDF |
| | | IPGVAIAANIIHEGFHKSRKVIVVVSQHFI |
| | | QSRWCIFEYEIAQTWQFLSSRAGIIFIVLQ |
| | | KVEKTLLRQQVELYRLLSRNTYLEWEDSVL |
| | | GRHIFWRRLRKALLDGKSWNPEGTVGTGCN |
| | | WQEATSI |
| 27 | TLR9 | EVQAAVPGLPSRVKCGSPGQLQGLSIFAQD |
| | | LRLCLDEALSWDCFALSLLAVALGLGVPML |
| | | HHLCGWDLWYCFHLCLAWLPWRGRQSGRDE |
| | | DALPYDAFVVFDKTQSAVADWVYNELRGQL |
| | | EECRGRWALRLCLEERDWLPGKTLFENLWA |
| | | SVYGSRKTLFVLAHTDRVSGLLRASFLLAQ |
| | | QRLLEDRKDVVVLVILSPDGRRSRYVRLRQ |
| | | RLCRQSVLLWPHQPSGQRSFWAQLGMALTR |
| | | DNHHFYNRNFCQGPTAE |

In some embodiments, a chimeric receptor of the present disclosure may comprise a CD3ζ signaling domain: (RVKFSRSADAPAYQQGQNQLYNELNLGRREEYDVLDKRRGRDPEMGGKPRRKNP QEGLYNELQKDKMAEAYSEIGMKGERRRGKGHDGLYQGLSTATKDTYDALHMQ ALPPR; SEQ ID NO:43).

D. Linker/Hinge

The disclosed chimeric receptors may optionally comprise a linker or hinge connecting the target binding domain with the transmembrane domain. Various linkers/hinges that are commonly utilized in known chimeric antigen receptors (CARs) may be used here. For examples, in some embodiments, a linker or hinge may comprise an IgG4 hinge or derivative thereof, an IgG2 hinge or derivative thereof, a CD28 hinge, or a CD8 hinge. Specific exemplary transmembrane domains are included in the table below, but are not intended to be limiting.

| SEQ | | |
|---|---|---|
| ID | | |
| NO: | Description | Sequence |
| 28 | G-Linker-1 | GGGGSGGGGSGGGGS |
| 29 | G-Linker-2 | GGGGSGGGGS |
| 30 | G-Linker-3 | GGGGS |
| 31 | G-Linker-4 | GGGSSGGGSG |
| 32 | IgG4 hinge-1 | ESKYGPPCPSCP |
| 33 | IgG4 hinge-2 | ESKYGPPCPPCP |
| 34 | IgG4 hinge | ESKYGPPCPPCPGGGSSGGGSG |
| | linker | |
| 35 | CD28 hinge | IEVMYPPPYLDNEKSNGTIIHVK |
| | | GKHLCPSPLFPGPSKP |
| 36 | CD8 hinge-1 | AKPTTTPAPRPPTPAPTIASQPL |
| | | SLRPEACRPAAGGAVHTRGLDFA |
| | | CD |
| 37 | CD8 hinge-2 | TTTPAPRPPTPAPTIASOPLSLR |
| | | PEACRPAAGGAVHTRGLDFACD |
| 38 | IgG4-1 | ESKYGPPCPPCPGGGSSGGGSGG |
| | | QPREPQVYTLPPSQEEMTKNQVS |
| | | LTCLVKGFYPSDIAVEWESNGQP |
| | | ENNYKTTPPVLDSDGSFFLYSRL |
| | | TVDKSRWQEGNVFSCSVMHEALH |
| | | NHYTQKSLSLSLGK |
| 39 | IgG4-2 | GQPREPQVYTLPPSQEEMTKNQV |
| | | SLTCLVKGFYPSDIAVEWESNGQ |
| | | PENNYKTTPPVLDSDGSFFLYSR |
| | | LTVDKSRWQEGNVFSCSVMHEAL |
| | | HNHYTQKSLSLSLGK |

E. Co-Stimulatory Domain

The disclosed chimeric receptors may optionally comprise a co-stimulatory domain between the transmembrane domain and the intracellular signaling domain. In general, human protein sequences are preferred for the purposes of a co-stimulatory domain of the present chimeric receptors. Various co-stimulatory domains that are commonly utilized in known chimeric antigen receptors (CARs) may be used here. For examples, in some embodiments, the co-stimulatory domain may comprise a portion of CD28, 4-1BB, CD3, CD27, ICOS, OX40, HVEM, CD30 and/or any other member of the family of T cell co-stimulatory molecules. Specific exemplary transmembrane domains are included in the table below, but are not intended to be limiting.

| SEQ ID | | |
|---|---|---|
| NO: | Description | Sequence |
| 40 | CD28 | RSKRSRLLHSDYMNMTPRRPG |
| | | PTRKHQYPYAPPRDFAAYRS |
| 41 | 4-1BB | KRGRKKLLYIFKQPFMRPVQT |
| | | TQEEDGCSCRFPEEEEGGCEL |
| 42 | OX40 | ALYLLRRDQRLPPDAHKPPGG |
| | | GSFRTPIQEEQADAHSTLAKI |

### Vectors for Expressing a Chimeric Receptor

For the purposes of the present disclosure, a nucleic acid sequence encoding a chimeric receptor may be comprised within an expression vector, which is capable of expressing the chimeric receptor in a target cell (e.g., a monocytic cell like a macrophage). More specifically, the expression vector may be used to express one or more chimeric receptors on the surface of the target cell. Such a vector may further comprise regulatory sequences, including for example, a promoter, operably linked to the coding sequence, an enhancer, and/or a ribosomal entry site. The vector may optionally further comprise a selectable marker sequence, for instance for propagation in in vitro bacterial or cell culture systems. In some embodiments, the selectable marker may be a truncated protein or peptide, such as a truncated CD19.

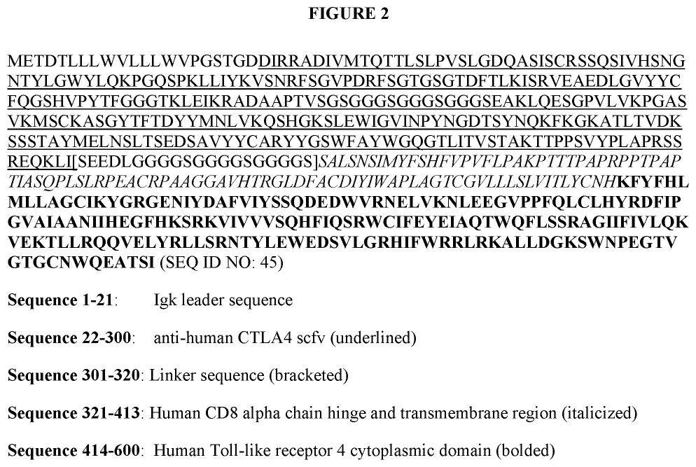

Preferred expression vectors may comprise one or more of an origin of replication, a suitable promoter and/or enhancer, and also any necessary ribosome binding sites, polyadenylation site, splice donor and acceptor sites, transcriptional termination sequences, and 5′ flanking nontranscribed sequences. DNA sequences derived from the SV40 or cytomegalovirus (CMV) viral genome, for example, SV40 origin, early promoter, enhancer, splice, and polyadenylation sites may be used to provide the required non-transcribed genetic elements. An exemplary expression vector is shown in FIG. 2. In some embodiments, the promoter may be a T7 promoter.

Specific initiation signals may also be required for efficient translation and expression of the chimeric receptor. These signals can include the ATG initiation codon and adjacent sequences. In some embodiments, an expression vector may comprise its own initiation codon and adjacent sequences may be inserted into the appropriate expression vector, and no additional translation control signals may be needed. However, in some embodiments, only a portion of an open reading frame (ORF) may be used, and exogenous translational control signals, including, for example, the ATG initiation codon, can be provided. Furthermore, the initiation codon may be in phase with the reading frame of the desired coding sequence (i.e., the nucleic acid sequence encoding the chimeric receptor) to ensure translation of the entire target sequence.

Exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:516-544 (1987)). Some appropriate expression vectors are described by Sambrook, et al., in Molecular Cloning: A Laboratory Manual, Second Edition, Cold Spring Harbor, N.Y. (1989), the disclosure of which is hereby incorporated by reference. If desired, to enhance expression and facilitate proper protein folding, the codon context and codon pairing of the sequence may be optimized, as explained by Hatfield et al., U.S. Pat. No. 5,082,767.

Promoters include, but are not limited to, EF-1a promoter, CMV immediate early, HSV thymidine kinase, early and late SV40, LTRs from retrovirus, and mouse metallothionein-I. Exemplary vectors include pWLneo, pSV2cat, pOG44, pXT1, pSG (Stratagene) pSVK3, pBPV, pMSG, and pSVL (Pharmacia). Selectable markers include CAT (chloramphenicol transferase). Preferred vectors also include cytoplasmic vectors, like the T7 vector system. See Wagner et al., U.S. Pat. No. 5,591,601 (Jan. 7, 1997).

In some embodiments, the vector may additionally comprise other functional sequence such as FSV non-structural protein genes and/or a FSV subgenomic promoter.

In some embodiments, the promoter may be inducible. Inducible promoters operably link the expression of target genes (e.g., a chimeric receptor) to a specific signal or a particular biotic or abiotic factor. Types of inducible promoters that may be utilized in the disclosed expression system include, but are not limited to, chemically-inducible promoters (i.e., antibiotics, steroids, metals, etc.), light-inducible promoters, heat-inducible promoters, and hypoxia-inducible promoters.

In some embodiments, transcription and expression of a chimeric receptor can be controlled by a hypoxia-inducible promoter. Transcriptional regulation of gene expression under hypoxia can be mediated by hypoxia induced factor 1 (HIF1). The binding of HIF1 to HIF1 responsive elements (FIRE) in an enhancer sequence of a promoter leads to gene expression. Several gene promoters have been found to be hypoxia-inducible including, but not limited to, erythropoietin gene, phosphoglycerate kinase-1, and VEGF (The Journal of Experimental Biology 201, 1153-1162, 1998).

Since native promoters are regulated by multiple transcription factors, it is also possible to make a chimeric promoter that is more specific to hypoxia (Gene Therapy (2002) 9, 1403-1411). Thus, in some embodiments, a chimeric promoter can be constructed with an enhancerless basal viral promoter, such as SV40 and CMV, and several copies of HRE. For example, in some embodiments, the disclosed expression system can comprise a chimeric promoter of HREx3+Basal SV40 promoter.

Incorporating a hypoxia-inducible promoter into an expression vector for expressing a chimeric receptor can increase tumor targeting by tumor associated macrophages (TAMs) that may have taken up the disclosed delivery vector. The microenvironment of the tumor is generally the only hypoxic environment in an otherwise healthy body, and TAMs may localize to a hypoxic tumor bed. Thus, if the disclosed delivery vectors are administered to a subject systemically (e.g., in proximity to a tumor-draining lymph node) and phagocytosed by monocytic cells in the lymph node or in circulation, the chimeric receptor encoded by the expression vector will not be expressed until the monocytic cell has infiltrated into the tumor bed and is exposed to hypoxic conditions. This will result in tumor targeted expression of the disclosed chimeric receptors.

In view of the foregoing, in some embodiments, the present disclosure provides delivery vectors and expression vectors for activating the immune system by engineering tumor-associated macrophages (TAMs) and other monocytic cells to express a chimeric receptor, such as a receptor comprising an immune checkpoint-specific target binding domain (e.g., an anti-CTLA-4 scFv) or an OX40-specific target binding domain (e.g., scFv mderived from 9B12, MOXR0916, PF-04518600, MEDI0562, MEDI6469, MEDI6383, PF-04518600, or BMS 986178), a transmembrane domain (e.g., a CD8 transmembrane domain), and an intracellular signaling domain (e.g., the intracellular signaling domain of a TLR, such as TLR4 or TLR9). Expression of the disclosed chimeric receptors by monocytic cells, either systemically (such as in a tumor adjacent lymph node) or specifically within a tumor bed, can competitively block binding of immune checkpoints, such as CTLA-4, PD-1, and PD-L1, and/or stimulate OX40 signaling, thereby activating the immune system to elicit a strong and tumor specific anti-tumor response that results in destruction of the tumor and treatment of the disease. Moreover, binding of the chimeric receptor to its target molecule (e.g., CTLA-4, PD-1, PD-L1, OX40, etc.) not only inhibits checkpoint signaling or agonizes OX40 signaling, but also stimulates the intracellular signaling domain of the chimeric receptor (i.e., the intracellular domain of a TLR). This can, for example, initiate a cytokine response that further attacks and destroys the tumor by creating an active immune environment and propagating an aggressive immune response. When various TLR domains, such as TLR4 and TLR9, are used as the intracellular signaling domain of the chimeric receptor, activation of the chimeric receptor by binding a target molecule will trigger expression of cytokines, including but not limited to, IL-12, IFN-α, IRN-γ, TNF-α, IL-6, and/or IL-1β, thereby overcoming or circumventing the immune suppressive environment of the tumor.

In some embodiments, monocytic cells may to exposed to, and therefore phagocytose, a delivery vector that comprises more than one expression vector, and the expression vectors may encode the same or different chimeric receptors. For example, a single deliver vector may comprise expression vectors that encode an anti-CTLA-4 chimeric receptor and an anti-PD-1 chimeric receptor. Additionally or alternatively, in some embodiments a given monocytic cell may be exposed to, and therefore phagocytose, more than one delivery vector, each of which comprises a different expression vector encoding a different chimeric receptor. For example, a monocytic cell may phagocytose two different deliver vectors, one of which comprises an expression vector that encodes an anti-CTLA-4 chimeric receptor and another of which comprises an expression vector that encodes an anti-PD-1 chimeric receptor. Accordingly, in some embodiments, the present disclosure provides monocytic cells (such as tumor associated macrophages) that express 1, 2, 3, 4, or 5 or more different chimeric receptors, as disclosed herein.

### Delivery Vectors

The present disclosure provides a solid matrix-based composition for directed entry into a monocyte cell (hereafter a “delivery vector”). A delivery vector according to the present disclosure is generally composed of a base particle that can be phagocytized by monocytic cells with a virus component attached to the surface of the base particle. The virus component can function not only to help evade the lysosome upon phagocytosis by a monocytic cell, but may also comprise an expression vector for encoding a chimeric receptor, as discussed above. The disclosed delivery vectors are highly specific for phagocytic cells like monocyte cells, including dendritic cells and macrophages. This pronounced selectivity for monocyte cells renders the delivery vectors extremely useful for gene therapy and other gene medicine methods requiring introduction and expression of genes (e.g., a gene encoding a chimeric receptor) into cells of the monocyte lineage.

A. Base Particle

The disclosed delivery vectors take advantage of the phagocytic activity of monocyte cells by “looking” like a bacterium. Thus, a preferred size for the base particle is one that approximates the size of the bacterial antigens that monocyte cells typically ingest. Generally, the vector particle will be about 0.5 to about 2.5 microns, or about 0.5 to about 1 micron. Thus, the vector particle may be about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, or about 2.5 microns.

In some embodiments, the base particle may be a yeast cell wall particle (YCWP), such as yeast glucan particles. In some embodiments, the base particle may be a bead.

i. Yeast Cell Wall Particle (YCWP)

A YCWP can be prepared from yeast cell wall such that the particle is porous to the delivery of various macromolecules. In one embodiment, the YCWP can be prepared from Saccharomyces cerevisiae. In another embodiment, the YCWP can a zymosan particle. In another embodiment, the YCWP approximates the size of microbial structures that cells of the mononuclear phagocyte system and other phagocytic cells typically ingests (e.g., bacteria). In specific embodiments, the YCWP can be about 1-5 μm.

In some embodiments, the YCWP may be prepared by (a) suspending yeast to produce a suspension, (b) incubating the suspension, (c) centrifuging the suspension and removing the supernatant and (d) recovering the resulting YCWP. In some embodiments, steps (a)-(d) are repeated at least 1, 2, 3 or 4 times.

In some embodiments, the YCWP may be prepared by (a) suspending yeast in a solution to produce a first suspension, (b) incubating the first suspension, (c) centrifuging the first suspension and removing the supernatant, (d) suspending the resulting pellet to produce a second suspension, (e) incubating the second suspension, (f) centrifuging the second suspension and removing the supernatant and (g) washing the resulting pellet to recover the YCWP. In some embodiments, the YCWP is sterilized.

In some embodiments, the yeast is suspended in NaOH, including 1M NaOH. In some embodiments, the first suspension is incubated at about 80° C. for about 1 hour or for 1 hour. In some embodiments, the centrifuging is performed at about 2000 times gravity for about 10 minutes, or at 2000 times gravity for 10 minutes. In some embodiments, the pellet is suspended in water, including water at about pH 4.5 or at pH 4.5. In some embodiments, the second suspension is incubated at about 55° C. for about 1 hour or at 55° C. for 1 hour. In some embodiments, the pellet is washed in water at least 1, 2, 3 or 4 times. In some embodiments, the pellet is washed once.

In some embodiments, the YCWP is sterilized using isopropanol and/or acetone following washing of the pellet. In specific embodiments, other known alcohols are appropriate. In some embodiments, the YCWP is allowed to fully dry after sterilization. In some embodiments, the YCWP is resuspended after being allowed to dry. In some embodiments, the YCWP is freeze dried and store at 4° C.

YCWP have a pore size of at least about 30 nm, and therefore, any molecule/object with a radius of rotation of 30 nm or less can be loaded within the yeast cell wall particles. For example, some viruses or viral particles having a size less than 30 nm (e.g., tobacco mosaic virus) can be loaded within yeast cell wall particles, as well as other antigens, including tumor lysate. When a YCWP is utilized as the base particle for the disclosed delivery vector, the anti-tumor activity of the delivery vector may be enhanced by loading the YCWP with an antigenic component, such as a tumor antigen or tumor cell lysate. This can add to or synergize the immune stimulation of the delivery vector beyond the expression of the chimeric receptor by allowing the delivery vector to simultaneously function as a tumor vaccine. Thus, in some embodiments, the YCWP is some in PBS, such as 1×PBS. In some embodiment, the YCWP is allowed to dry and then frozen before the tumor lysate is loaded into the YCWP, in order to place it in storage before use. In some embodiments, the YCWP is freeze dried and store at about 4° C. or lower.

“Tumor lysate” refers to a solution produced when the cell membranes of tumor cells are disrupted, either by physical or chemical methods. In some embodiments, tumor lysate is prepared from a solid tumor including, but not limited to carcinomas and sarcomas. In some embodiments, tumor lysate is prepared from a tumor cell line. In some embodiments, tumor lysate is prepared from any solid tumor or tumor cell lines relating to breast cancer, small cell lung cancer, non-small cell lung cancer, glioma, medulloblastoma, neuroblastoma, Wilms tumors, rhabdomyosarcoma, osteosarcoma, liver cancer, pancreatic cancer, melanoma, prostate cancer and ocular melanoma. In some embodiments, tumor lysate is produced under a number of conditions, including repeated freezing and thawing, homogenizing, contacting with a hyper- or hypo-tonic solution or contacting with one or more non-ionic detergents.

YWCPs may be loaded with a biological material, such as a specific protein or a fragment thereof, nucleic acid, carbohydrate, tumor lysate, or a combination thereof. In some embodiments, the biological material can be loaded into the YCWP by incubating the biological material and a suspension of YCWP together and allowing the biological material to penetrate into the hollow insides of the particles.

In some embodiments, after the YCWP is incubated or loaded with the biological material, the combination is freeze-dried to create an anhydrous particle. By freeze-drying, the biological material is trapped within the particle. In some embodiments, the freeze-drying is the only mechanism used to trap the biological material within the particle. In some embodiments, the entrapment is not caused by a separate component blocking the biological material from exiting the particle, for example, by physical entrapment, hydrophobic binding, any other binding. In some embodiments, the entrapment is not caused by crosslinking or otherwise attaching the biological material to the particle outside of any attachment that may occur upon freeze-drying. In some embodiments, the compositions of the present invention do not include any additional component that specifically assists in evading the lysosome. The biological material includes, for example, a specific protein or a fragment thereof, nucleic acid, carbohydrate, tumor lysate, or a combination thereof. In some embodiments, the number of YCWPs is about 1×10⁹ and the volume of biological material is about 50 μL. In specific embodiments, the incubation is for about one hour or less than one hour at about 4° C. In some embodiments, the combination of YCWPs and biological material is freeze dried over a period of less than or about 2 hours.

In some embodiments, the biological material is loaded into the particle by (a) incubating the biological material and a suspension of the YCWPs, allowing the biological material to penetrate into the hollow insides of the particles and freeze-drying the suspension of loaded particle and (b) optionally resuspending the particles, incubating the resuspended particles and freeze drying the resuspended particles.

In some embodiments using YCWPs, the number of YCWPs is about 1×10⁹ and the volume of the biological material is about 50 μL. In some embodiments, the number of YCWPs is 1×10⁹ and the volume of the biological material is 50 μL. In some embodiments, the incubation in step (a) is for less than one hour at about 4° C. In specific embodiments, the incubation in step (a) is for about one hour at 4° C. In some embodiments, the foregoing suspension is freeze dried in step (a) over a period of less than 2 hours or over a period of about 2 hours. In some embodiments, the YCWPs in step (b) are resuspended in water, including about 50 μL of water or 50 μL of water. In some embodiments, the resuspended YCWPs are incubated in step (b) for less than or about one hour at about 4° C. or for less than or about 2 hours at 4° C. The biological material includes a specific protein or a fragment thereof, nucleic acid, carbohydrate, tumor lysate, or a combination thereof.

In some embodiments, the loaded YCWP is coated with a silicate. Specifically, in some embodiments the loaded YCWPs are coated by contacting the YCWPs with a silicate, such as tetraalkylorthosilicate, in the presence of ammonia, such that the loaded YCWPs are capped with the silicate. In preferred embodiments, the loaded YCWPs are capped with the silicate within about 60 minutes, about 45 minutes, about 30 minutes, about 15 minutes, about 10 minutes, about 5 minutes or about 2 minutes. The reactivity of the tetraalkylorthosilicates is such that under hydrolysis mediated by the ammonia, the tetraalkylorthosilicates react with the primary hydroxyls of the β-glucan structure of the YCWPs. The tetraalkylorthosilicates also self-react with the ends of these cell wall silicates to form “bridges” such as —O—Si(OH)₂—O— or in three dimensions such as —O—Si(—O—Si—O—) (OH)—O— or —Si(—O—Si—O—)₂—O—. These bridges may occur across the pores in the YCWPs such that the retention of the loaded drug or biological material therein is increased. Such a capped, loaded YCWP can be freeze dried.

ii. Bead Particle

In those embodiments when a bead is used as the base particle, several factors must be weighed to determine the ideal bead for a given need. For instance, from the perspective of uptake, the smaller end of the ranges is preferred, because it more closely approximate the size of a bacterium. On the other hand, for manufacturing purposes, slightly larger particles may be preferred, because they may be less likely to stick together.

Furthermore, the bead particle is not limited by shape or material. The bead particle can be of any shape, size, or material that allows the bead vector to be phagocytized by monocytic cells.

In some embodiments, the base particle be selected from any known ferro-magnetic center covered by a polymer coat. For example, beads that can be used as a base particle include, but are not limited to, microbeads, microspheres, and silicate beads. Such beads may be preferred in certain applications because magnetic separation can be employed to separate free from bead-bound components during processing. However, bead particles for use in the disclosed delivery vectors are not limited to a specific type of material and may be made of synthetic materials like polystyrene or other plastics, as well as biological materials.

B. Virus Component

In addition to the base particle, a delivery vector of the present disclosure may also comprise a virus component, for example, a retrovirus or adenovirus attached or conjugated to the base particle. The role of the virus component with respect to the delivery vector is to assist the vector in escaping the harsh environment of the lysosome following phagocytosis by a monocyte cell and to deliver the nucleic acid or expression construct that encodes a chimeric receptor for expression on the surface of the target monocytic cell.

When a monocytic cell ingests a large antigen, a phagocytic vesicle (phagasome) is formed which engulfs the antigen. Next, a specialized lysosome contained in the monocyte cell fuses with the newly formed phagosome. Upon fusion, the phagocytized antigen is exposed to several highly reactive molecules as well as a concentrated mixture of lysosomal hydrolases. These highly reactive molecules and lysosomal hydrolases digest the contents of the phagosome. By attaching a virus component to the particle, the nucleic acid that is contained within the virus can escape digestion by the materials in the lysosome and enters the cytoplasm of the monocyte intact. Prior systems have failed to recognize the importance of this feature and, thus, obtained much lower levels of expression than the expression systems of the present disclosure. See Falo et al., WO 97/11605 (1997).

Thus, the present disclosure provides a delivery vector in which one or more viruses capable of expressing a chimeric receptor in a target cell (e.g., a monocytic cell) are attached to the surface of a base particle (e.g., a YCWP or bead particle). The virus may be an RNA virus, like a retrovirus, or a DNA virus, like an adenovirus. In some embodiments, the virus may be recombinant and/or non-replicative and/or non-infective. One of skill in the art will know of commonly used methods to make a virus non-replicative and/or non-infective.

In some embodiments, the virus itself may be is capable of lysosome disruption. Alternatively, the virus may not be capable of lysosome disruption. In such a case, a separate lysosome evading component may be added. Preferred viruses include adenovirus (e.g., Ad5), lentivirus (e.g., HIV-derived viruses), and adeno associate virus (“AAV”; e.g., AAV5, AAV9, etc.).

A single base particle may have numerous virus components attached or conjugated to its surface. Each virus component may encode a single chimeric receptor or more than one (e.g., 2, 3, 4, 5, or more) chimeric receptors. Thus, in some embodiments, a base particle may have multiple virus components, each encoding a different chimeric receptor, attached or conjugated to its surface. A monocytic cell that phagocytoses such a delivery vector would therefore be able to express multiple different chimeric receptors on its surface, for example, one chimeric receptor that specifically binds CTLA-4 and one chimeric receptor that specifically binds PD-1 or PD-L1. Accordingly, in some embodiments, the present disclosure provides monocytic cells (such as tumor associated macrophages) that express 1, 2, 3, 4, or 5 or more different chimeric receptors, as disclosed herein.

Because viral infection is not essential for the nucleic acid or expression vector encoding a chimeric receptor to reach the cytoplasm of the monocyte cell, the virus can also be replication/infection deficient. For example, one method for producing a replication/infection deficient adenovirus can be achieved by altering the virus fiber protein. Thus, in some embodiments, a virus in which the fiber protein is engineered by specific mutations to allow the fiber protein to bind to an antibody but not to its cognate cellular receptor can be used in the particles of the present disclosure.

Another method for producing a replication/infection deficient virus is by intentionally causing denaturation of the viral component responsible for infectivity. In the case of adenovirus, for example, the fiber protein could be disrupted during the preparation of the virus. For HIV, this could include the envelope (env) protein. Thus, in some embodiments, a method for creating an infection deficient virus for attachment to the disclosed bead particles comprises removing the outer membranes of the virus so that only the virus core remains.

In some embodiments, it may be beneficial for the expression vector encoding the chimeric receptor to stably integrate into the target cell chromosome. For example, one mode for achieving stable integration is through the use of an adenovirus hybrid. Such an adenovirus hybrid may comprise, for example, an adenoviral vector carrying retrovirus 5′ and 3′ long terminal repeat (LTR) sequences flanking the nucleic acid component encoding a chimeric receptor and a retrovirus integrase gene (see Zheng, et al. Nature Biotechnology, 18:176-180, 2000).

In some embodiments, transient expression may be preferred and cytoplasmic viruses, like Sindbis virus, for example, can therefore be employed.

In some embodiments, where no lysosome evading component is naturally present on the virus, one may be added. For example, in the case of Sindbis or other such viruses, the virus can be engineered to express all or part of the adenovirus penton protein for the purpose of evading the lysosome.

In some embodiments, the disclosed delivery vectors may comprise a further lysosome evading component that is capable of evading or disrupting the lysosome attached to the base particle. For example, such a lysosome evading component can include proteins, carbohydrates, lipids, fatty acids, biomimetic polymers, microorganisms and combinations thereof. It is noted that the term “protein” encompasses a polymeric molecule comprising any number of amino acids. Therefore, a person of ordinary skill in the art would know that “protein” encompasses a peptide, which is understood generally to be a “short” protein. In some embodiments, lysosome evading components include, but are not limited to, specific viral proteins. For example, the adenovirus penton protein is a complex that enables a virus to evade/disrupt the lysosome/phagosome. Thus, either the intact adenovirus or the isolated penton protein, or a portion thereof (see, e.g., Bal et al., Eur J Biochem 267:6074-81 (2000)), can be utilized as the lysosome evading component. In some embodiments, fusogenic peptides derived from N-terminal sequences of the influenza virus hemagglutinin subunit HA-2 may also be used as the lysosome evading component (Wagner, et al., Proc. Natl. Acad. Sci. USA, 89:7934-7938, 1992).

Other lysosome evading components include, but are not limited to, biomimetic polymers such as Poly (2-propyl acrylic acid) (PPAAc), which has been shown to enhance cell transfection efficiency due to enhancement of the endosomal release of a conjugate containing a plasmid of interest (see Lackey et al., Abstracts of Scientific Presentations: The Third Annual Meeting of the American Society of Gene Therapy, Abstract No. 33, May 31, 2000-Jun. 4, 2000, Denver, Colo.) Examples of other lysosome evading components envisioned by the present invention are discussed by Stayton, et al. J. Control Release, 1; 65(1-2):203-20, 2000.

Viruses can be attached to the base particles directly, using conventional methods, or indirectly. See Hammond et al., Virology 254:37-49 (1999). For example, YCWPs can be oxidized with sodium periodate to generate aldehydes, which can be further reacted with adipic acid dihydrazide to form ADH-particles. These ADH-particles can be derivatized with SPDP (succinimidyl 3-(2-pyridyldithio)propionate) crosslinker and reacted with SPDS derivatized avidin to form YCWP conjugated with Avidin, i.e., avidin-modified YCWPs. Avidin-modified YCWPs can be directly used for conjugation of biotinylated viral particles (e.g., biotinylated adenovirus) or they can be further modified with Biotin-polyethyleneimine (PEI). Avidin-modified YCWPs can be saturated with PEI-g-PEG-Biotin to form PEI modified particles, PEI-particles. Adenovirus (and other anionic viruses) can be carried by PEI-particles through charge interactions between the PEI and the anionic charge of the virus coat.

Thus, in some embodiments, the target nucleic acids may be delivered in a recombinant adenovirus that is conjugated to the base particle via a biotin-streptavidin linkage. The base particle may be modified to attach a linker comprising streptavidin and the recombinant virus may be biotinylated.

Other processes or mechanisms may also be used to attach the virus component to the base particle. For example, antibody attachment may be a similarly efficient way to attach a desired virus to the base particle. One example of antibody attachment encompassed may comprise a single antibody that is chemically affixed to the bead vector particle. The antibody is specific to the component to be attached to the base particle.

Alternatively, two or more antibodies can be used. In this case, one antibody, which attached to the base particle, may be specific for a second antibody. The second antibody is specific to the virus to be attached to the base particle. Thus, the virus-specific antibody binds the virus, and that antibody, in turn, is bound by the base particle-bound antibody. For instance, a goat- or rabbit-anti-mouse antibody may be bound to the bead and a mouse monoclonal antibody used to bind the specific virus. Or, in another alternative format, the two or more antibodies my each be specific for a different virus to be attached to the particle, such that the particle is decorated with two or more distinct components (i.e., two distinct viral particles)

In another example of antibody attachment, protein A, or any similar molecule with an affinity for antibodies, is employed. In this example, the base particle may be coated with protein A, which binds to an antibody, and, in turn is bound to the virus being attached to the base particle.

In some embodiments, attaching viruses to a base particle can also be accomplished by engineering the virus to express certain proteins on its surface. For instance, the HIV env protein might be replaced with the adenovirus penton protein, or a portion thereof. The recombinant virus then could be attached via an anti-penton antibody, with attachment to the base particle mediated, for example, by another antibody or protein A. In some embodiments, a penton protein may also serve as a lysosome evading component.

### Pharmaceutical Compositions

Pharmaceutical compositions suitable for use in the methods described herein can include the disclosed delivery vectors and a pharmaceutically acceptable carrier or diluent.

The composition may be formulated for intradermal, intravenous, intratumoral, subcutaneous, intraperitoneal, intramuscular, oral, nasal, pulmonary, ocular, vaginal, or rectal administration. In some embodiments, the disclosed delivery vectors are formulated for intradermal, intravenous, subcutaneous, intraperitoneal, or intramuscular administration, such as in a solution, suspension, emulsion, etc. In some embodiments, the disclosed delivery vectors are formulated for oral administration, such as in a tablet, capsule, powder, granules, or liquid suitable for oral administration.

In some embodiments, the disclosed delivery vectors may be formulated for parenteral administration by, for example, intradermal, intravenous, intramuscular or subcutaneous injection. Formulations for injection may be presented in unit dosage form, e.g., in ampules or in multi-dose containers, optionally with an added preservative. The compositions may take such forms as suspensions, solutions or emulsions in oily or aqueous vehicles, and may contain formulatory agents such as suspending, stabilizing and/or dispersing agents. The delivery vector may also be formulated using a pharmaceutically acceptable excipient. Such excipients are well known in the art, but typically will be a physiologically tolerable aqueous solution. Physiologically tolerable solutions are those which are essentially non-toxic. Preferred excipients will either be inert or enhancing.

In some embodiments, the delivery vectors may be formulated to be administered concurrently with another therapeutic agent. In some embodiments, the delivery vectors may be formulated to be administered in sequence with another therapeutic agent. For example, the delivery vectors may be administered either before or after the subject has received a regimen of chemotherapy.

### Methods of Treatment

Provided herein are methods of treating tumors, cancer, malignant disease, or cancer cell proliferation with the disclosed delivery vectors. More specifically, the disclosure provides for methods of stimulating the immune system to mount an anti-tumor or anti-cancer response through the expression of a chimeric receptor on a monocytic cell. The mechanism of immune stimulation may be multi-faceted and may vary depending on the components of the chimeric receptor and whether the base particle of the delivery vector is concurrently loaded with a biological material for stimulating an immune response, such as a tumor lysate. The immune response may also vary depending on the specificity of the chimeric receptor (i.e., whether the receptor specifically binds CTLA-4, PD-1, PD-L1, OX40, or any other target disclosed herein) and how many distinct chimeric receptors are expressed on a given monocytic cell, as the disclosed delivery vectors can be utilized to express multiple distinct chimeric receptors on a single monocytic cell.

In some embodiments, the disclosed delivery vectors may provide to a monocytic cell, such as a macrophage or dendritic cell, a nucleic acid sequence and/or expression vector that encodes at least one chimeric receptor capable of specifically binding to an immune checkpoint protein, such as CTLA-4, PD-1, PD-L1, OX40, or any of the other target proteins disclosed in Table 1. In some embodiment, the disclosed delivery vectors may provide to a monocytic cell 1, 2, 3, 4, or 5 or more nucleic acid sequences and/or expression vectors that encode 1, 2, 3, 4, or 5 or more different chimeric receptors, which possess different target specificities and/or different intracellular TLR domains. When such a chimeric receptor is expressed on the surface of a monocytic cell, such as a tumor associated macrophage (TAM), the cell itself may function as a checkpoint inhibitor by binding the target protein and preventing signaling that would otherwise downregulate the tumor immune response.

For example, CTLA-4, also known as CD152 (cluster of differentiation 152), is a protein receptor that downregulates immune responses by functioning as an immune checkpoint. CTLA-4 is constitutively expressed on Tregs but only upregulated in conventional T cells after activation. It acts as an “off” switch when bound to CD80 or CD86 on the surface of antigen-presenting cells. CTLA-4 is homologous to the T-cell co-stimulatory protein, CD28, and both molecules bind to CD80 and CD86, also called B7-1 and B7-2 respectively, on antigen-presenting cells. CTLA-4 binds CD80 and CD86 with greater affinity and avidity than CD28 thus enabling it to outcompete CD28 for its ligands. CTLA-4 transmits an inhibitory signal to T cells, whereas CD28 transmits a stimulatory signal. CTLA-4 is also found in regulatory T cells and contributes to its inhibitory function. T cell activation through the T cell receptor and CD28 leads to increased expression of CTLA-4.

The mechanism by which CTLA-4 acts on T cells remains somewhat controversial. Biochemical evidence suggests that CTLA-4 recruits a phosphatase to the T cell receptor (TCR), thus attenuating the signal. More recent work has suggested that CTLA-4 may function in vivo by capturing and removing B7-1 and B7-2 from the membranes of antigen-presenting cells, thus making these unavailable for triggering of CD28. Expression of the disclosed chimeric receptors on monocytic cells, specifically monocytic cells within the tumor bed like TAMs, may bind up CTLA-4, allowing CD28 signaling to propagate and stimulate the immune system. This is, of course, only one example, and similar results may be achieved by targeting an alternative immune checkpoint like PD-1 or PD-L1.

Thus, the present disclosure provides methods for activating the immune system by engineering tumor-associated macrophages (TAMs) to express a chimeric receptor capable of binding and/or inhibiting an immune checkpoint. Expression of the disclosed receptors within a tumor bed or in a tumor adjacent lymph node (such as a tumor-draining lymph node) can competitively block checkpoint signaling and elicit a strong and tumor-specific intra-tumor checkpoint inhibition that results in destruction of the tumor and treatment of the disease.

This unique mechanism of action is a dramatic improvement over the current state of checkpoint inhibiting therapeutics. Currently, checkpoint inhibitors such as Pembrolizumab (Keytruda), Nivolumab (Opdivo), Atezolizumab (Tecentriq), and Ipilimumab (Yervoy) are effective at treating various types of cancer. However, these drugs are administered systemically, and therefore, cause off-target effects that can be life threatening. Indeed, checkpoint inhibitors are known to cause a unique spectrum of side effects termed immune-related adverse events (irAEs), which can include dermatologic, gastrointestinal, hepatic, endocrine, and other organ system effects. The disclosed delivery vectors prevent or minimize these off-target effects by expressing the encoded chimeric receptor only within or nearby the tumor microenvironment, thus decreasing side effects through cell-based tumor targeting.

In those embodiments in which an OX40 agonist (e.g., an extracellular domain of OX40L or an anti-OX40 antibody like 9B12, MOXR0916, PF-04518600, MEDI0562, MEDI6469, MEDI6383, PF-04518600, or BMS 986178) is used as the target binding domain, the chimeric receptor will modulate T cell activation and Tell effector function. Agonizing OX40 will enable effector T cells to survive and continue proliferating over an extended period of time, predominantly by transmitting anti-apoptotic signals that prevent excessive T cell death. This ultimately results in greater numbers of T cells surviving the primary immune response and developing into memory T cells that can then respond in secondary immune reactions when an antigen is reencountered at a later time. Moreover, preclinical studies have also shown that OX40 agonists may exert additional anticancer activity by depleting the number of FoxP3+ regulatory T (Treg) cells, which express high levels of OX40. Thus, OX40 is a particularly attractive target molecule for the disclosed chimeric receptors to bind.

In addition to the checkpoint inhibition mechanism of action and/or OX40 agonist mechanism of actions discussed above, the disclosed monocytic cells expressing a chimeric receptor may further elicit immune activation and an anti-tumor response by stimulating cytokine expression. For example, in some embodiments, the chimeric receptors of the disclosure comprise a TLR intracellular signaling domain, such as the intracellular signaling domain of TLR4 (Ref. Seq. NP_003257, NP_612564, or NP_612567; UniProt 000206; Entrez 7099) or TLR9 (Ref. Seq. NP_059138; UniProt Q9NR96; Entrez 54106). Binding of the chimeric receptor to its target molecule (e.g., CTLA-4, PD-1, PD-L1, OX40, etc.) will activate the intracellular domain, meaning that when the intracellular domain of a TLR is used, target binding will trigger a signaling cascade that leads to a pro-inflammatory cytokine response. The precise cytokine response will depend on the TLR domain that is used. In some embodiments, the chimeric receptor will be designed to trigger expression of M1-type cytokines, such as IL-12, IFN-α, TNF-α, IL-6, and/or IL-1β, in order to mount an aggressive, anti-tumor immune response and overcome or circumvent the immune suppressive and/or immune evasive signals that usually typify a tumor microenvironment. This mechanism is distinct from other cell-based therapy approaches, such as CAR T-cells, because the tumor/cancer is not directly attacked or phagocytosed, but instead it is destroyed by creating and propagating a milieu around the tumor that is immune active. As a result of this novel mechanism, a cancer or tumor may be treated even when the chimeric receptor-expressing cells are not in direct contact with the tumor or tumor cells. For instance, localization of the chimeric receptor-expressing cells in a tumor adjacent lymph node, such as a tumor-draining lymph node, will be sufficient to activate the immune system to destroy the tumor. Thus, monocytic cells expressing a checkpoint-specific chimeric receptor, as disclosed herein, may provide anti-tumor benefits in at least two unique ways.

Furthermore, when the disclosed delivery vector comprises a YCWP as the base particle, the vector can possess even further anti-tumor activity by loading the YCWP with a biological material, such as a tumor lysate. Inclusion of a biological material like a tumor lysate within the YCWP provides a vaccine-like function when the delivery vectors are taken up by an antigen presenting cell (APC) like cells of the mononuclear phagocyte system, including monocytes, macrophages, dendritic cells or immature dendritic cells. In the field of vaccination, cells of the mononuclear phagocyte system are considered “professional” antigen presenting cells and thus, are the ideal target for vaccine delivery. It is well known that presentation of an antigen within an APC is vastly more effective in generating a strong cellular immune response than expression of this same antigen within any other cell type. Accordingly, loading the YWCP with an antigenic biological material like a tumor lysate will result in the presentation of a tumor antigen on an antigen presenting cell via class I MHC and class II MHC molecules, thus dramatically enhancing the immune response elicited by the disclosed delivery vectors.

Due to the constant infiltration of new macrophages into the tumor bed, the disclosed delivery vectors may produce these improved effects when they are administered intradermally, subcutaneously, systemically (e.g., parenterally), or by directly injecting them into the tumor or a target lymph node (i.e., a tumor draining lymph node, such as the lymph node that an oncologist would assess for signs of metastasis). In some embodiments, the disclosed delivery vectors are injected intradermally into the skin near a target lymph node, as this may lead to the greatest amount of uptake by phagocytic monocytes.

The disclosed delivery vectors are highly selective for monocyte cells (e.g., macrophages, dendritic cells, or TAMs). It is, therefore, useful for any application involving selectively introducing an expression into a monocyte cell. In some embodiments, the disclosed vectors are administered to treat cancer, and, in particular, solid tumors. In view of the foregoing explanation of the putative mechanism of action, it is believed that the disclosed delivery vectors may be used to treat almost any type of cancer, particularly cancers comprising at least one solid tumor, which may include but is not limited to breast cancer, small cell lung cancer, non-small cell lung cancer, glioma, medulloblastoma, neuroblastoma, Wilms tumors, rhabdomyosarcoma, osteosarcoma, liver cancer, pancreatic cancer, melanoma, prostate cancer, colon cancer, bladder cancer, head and neck cancers, esophageal cancer, and ocular melanoma. Typical methods comprise contacting a monocytic cell with a delivery vector, such that it is phagocytosed by the monocytic cell and the chimeric receptor is subsequently expressed on the surface of the cell.

As noted above, the delivery vectors may be injected directly into a tumor or they may be administered, intradermally, subcutaneously, or systemically (i.e., into the peritoneal of the subject). In some embodiments, the delivery vectors may be administered intradermally proximate to tumor or tumor-draining lymph node. There is a constant influx of macrophages into solid tumors, and therefore even macrophages that phagocytose the delivery particles systemically may still infiltrate the tumor bed and function to treat the tumor or prevent tumor growth. Moreover, in some embodiments, expression of the chimeric receptor may be under the control of a hypoxia-induced promoter, in which case the chimeric receptor will only be expressed once the monocytic cell that phagocytosed the delivery vector has infiltrated the tumor bed.

Alternatively or additionally, the delivery vectors may function once phagocytosed by macrophages by being expressed in a lymph node in proximity to the tumor or cancer that is to be treated. In these embodiments, the disclosed delivery vector may be administered intradermally or subcutaneously in an area proximate to the closest lymph node (e.g., the “target lymph node”) to the tumor that is targeted for treatment. In this sense, administration proximate to the target lymph node means into or as close to the target lymph node as possible, but at least closer to the target lymph node than any other lymph node. Once in the target lymph node, the macrophages that phagocytosed the delivery vectors will express the chimeric receptor. In this way, the disclosed delivery vectors and methods can be used to modify the genetic makeup of a target lymph node, which will aid in activating the anti-tumor immune response and localizing the response to the tumor site.

In some embodiments a monocyte cell may be contacted with the disclosed delivery vector either in vivo or in vitro. Hence, both in vivo and ex vivo methods of treatment are contemplated herein. Prior methods that targeted monocytic cells rely principally on isolated a patient's monocytic cells and manipulating them in vitro and then returning the cells to the patient. While such embodiments are contemplated in the present disclosure, the disclosed delivery vectors provide a substantial improvement because they may be used in both in vivo and ex vivo methods. Moreover, altering the route of administration can alter the monocytic cells targeted. For example, in the case of intravenous injection, macrophages may be targeted, and in the case of subcutaneous injection, dendritic cells may be targeted, while in cases of intradermal administration near a tumor-draining lymph node, TAMs may be targeted.

In some embodiments, in vivo methods comprise administering a delivery vector parenterally, for example, intravenously, intramuscularly, subcutaneously or intradermally, preferably in proximity to a target lymph node.

In some embodiments, ex vivo methods comprise contacting monocytic cells outside the body and then administering the contacted cells to a patient in need thereof. The cells may also be administered parenterally, for instance, via infusion. Monocytic cells that are contacted by delivery vectors in ex vivo methods may be autologous or allogeneic. Monocytic cells for use in ex vivo methods may be isolated by known methods of leukapheresis from a donor or from the patient (i.e., the ultimate recipient of the monocytic cells to be contacted with the disclosed bead vectors).

It is known that as tumors (both primary tumors and metastases alike) grow beyond a few millimeters in diameter and become deficient in oxygen, creating a hypoxic microenvironment within the tumor. When such tumors become oxygen starved, they secrete signal proteins, such as angiogenic factors to increase the blood supply into the hypoxic areas of the tumor.

As a part of the mechanism of angiogenic induction, hypoxic tumors secrete a signaling chemokine protein that attracts monocytes to the tumor. Monocytes attracted to the sites of growing tumors then become macrophages and assist in the induction of tumor angiogenesis. Therefore, an effective method of tumor targeting involves administering a therapeutically effective amount of a delivery vector encoding a chimeric receptor to a cancer patient, either directly or via ex vivo contact with monocytic cells. The monocyte cells containing the phagocytized delivery vector are attracted to the tumor site and, if the expression in under the control of a hypoxia-inducible promoter, will selectively express the chimeric receptor in the tumor microenvironment.

In some embodiments, administration of a delivery vectors will result in expression of an anti-checkpoint chimeric receptor by tumor-associated macrophages. For example, the TAMs that have phagocytosed the delivery vectors will express an anti-CTLA-4, anti-PD-1 and/or an anti-PD-L1 chimeric receptor in the tumor microenvironment, resulting in the inhibition of CTLA-4 and/or PD-1 checkpoint signaling.

In some embodiments, the tumor or cancer being treated includes, but is not limited to, a neurological cancer, breast cancer, a gastrointestinal cancer (e.g., colon cancer), renal cell carcinoma (e.g., clear cell renal cell carcinoma), or a genitourinary cancer (e.g., ovarian cancer). In some embodiments, the cancer is melanoma, lung cancer (e.g., non-small cell lung cancer), head and neck cancer, liver cancer, pancreatic cancer, bone cancer, prostate cancer, bladder cancer, or a vascular cancer. Indeed, the disclosed methods provide a broad spectrum approach to treating tumors, cancer, malignant disease, or cancer cell proliferation, so the type of disease to be treated is not particularly limited.

Dosage regimens can be adjusted to provide the optimum desired response (e.g., a therapeutic response like tumor regression or remission). For example, in some embodiments, a single bolus of delivery vectors may be administered, while in some embodiments, several divided doses may be administered over time or the dose may be proportionally reduced or increased as indicated by the situation. For example, in some embodiments the disclosed delivery vectors may be administered once or twice weekly by subcutaneous or intradermal injection. In some embodiments, the disclosed delivery vectors may be administered once or twice monthly by intradermal injection. In some embodiments, the disclosed delivery vectors may be administered once every week, once every other week, once every three weeks, once every four weeks, once every other month, once every three months, once every four months, once every five months, or once every six months.

Doses may likewise by adjusted to provide the optimum desired response (e.g., a therapeutic response like tumor regression or remission). For example, in some embodiments, a dose of the disclosed delivery vectors may comprise 1.0×10⁸ to 1.0×10¹² vectors. For example, a single dose may comprise 1.0×10⁸, 1.5×10⁸, 2.0×10⁸, 2.5×10⁸, 3.0×10⁸, 3.5×10⁸, 4.0×10⁸, 4.5×10⁸ 5.0×10⁸, 5.5×10⁸, 6.0×10⁸, 6.5×10⁸, 7.0×10⁸, 7.5×10⁸, 8.0×10⁸, 8.5×10⁸, 9.0×10⁸ 9.5×10⁸, 1.0×10⁹, 1.5×10⁹, 2.0×10⁹, 2.5×10⁹, 3.0×10⁹, 3.5×10⁹, 4.0×10⁹, 4.5×10⁹ 5.0×10⁹, 5.5×10⁹, 6.0×10⁹, 6.5×10⁹, 7.0×10⁹, 7.5×10⁹, 8.0×10⁹, 8.5×10⁹, 9.0×10⁹, 9.5×10⁹, 1.0×10¹⁰, 1.5×10¹⁰, 2.0×10¹⁰, 2.5×10¹⁰, 3.0×10¹⁰, 3.5×10¹⁰, 4.0×10¹⁰, 4.5×10¹⁰, 5.0×10¹⁰, 5.5×10¹° 6.0×10¹⁰, 6.5×10¹⁰, 7.0×10¹⁰, 7.5×10¹⁰, 8.0×10¹⁰, 8.5×10¹⁰, 9.0×10¹⁰, 9.5×10¹° 1.0×10¹¹, 1.5×10¹¹, 2.0×10¹¹, 2.5×10¹¹, 3.0×10¹¹, 3.5×10¹¹, 4.0×10¹¹, 4.5×10¹¹ 5.0×10¹¹, 5.5×10¹¹, 6.0×10¹¹, 6.5×10¹¹, 7.0×10¹¹, 7.5×10¹¹, 8.0×10¹¹, 8.5×10¹¹ 9.0×10¹¹, 9.5×10¹¹, or 1.0×10¹² vectors. In some embodiments, the dose may be about 9.5×10⁸, about 9.75×10⁸, about 9.85×10⁸, about 9.95×10⁸, about 1.0×10⁹, about 1.1×10⁹, about 1.15×10⁹, about 1.2×10⁹, about 1.25×10⁹, about 1.3×10⁹, about 1.35×10⁹, about 1.4×10⁹, about 1.45×10⁹, or about 1.5×10⁹ vectors.

Furthermore, the disclosed methods of treatment can additionally comprise the administration of a second therapeutic compound in addition to disclosed bead vectors. For example, in some embodiments, the additional therapeutic compound may be a CAR-T cell, a tumor-targeting antibody, an immune response potentiating modality, a checkpoint inhibitor, or a small molecule drug, such as a BTK inhibitor (e.g. ibrutinib), an EGFR inhibitor (e.g. CK-101), a BET inhibitor (e.g. CK-103), a PARP inhibitor (e.g. olaparib or CK-102), a PI3Kdelta inhibitor (e.g. TGR-1202), a BRAF inhibitor (e.g. Vemurafenib), or other chemotherapeutics known in the art.

Particular treatment regimens may be evaluated according to whether they will improve a given patient's outcome, meaning the treatment will reduce the risk of recurrence or increase the likelihood of progression-free survival of the given cancer or tumor.

Thus, for the purposes of this disclosure, a subject is treated if one or more beneficial or desired results, including desirable clinical results, are obtained. For example, beneficial or desired clinical results include, but are not limited to, one or more of the following: decreasing one or more symptoms resulting from the disease, increasing the quality of life of those suffering from the disease, decreasing the dose of other medications required to treat the disease, delaying the progression of the disease, and/or prolonging survival of individuals.

Furthermore, while the subject of the methods is generally a cancer patient, the age of the patient is not limited. The disclosed methods are useful for treating tumors, cancer, malignant disease, or cancer cell proliferation with various recurrence and prognostic outcomes across all age groups and cohorts. Thus, in some embodiments, the subject may be a pediatric subject, while in other embodiments, the subject may be an adult subject.

The following examples are given to illustrate the present disclosure. It should be understood that the invention is not to be limited to the specific conditions or details described in these examples.

### EXAMPLES

### Example 1—Preparation of Lentivirus

In various embodiments of the disclosed particles, the non-infective virus attached to the base particle may be a lentivirus. However, other non-infective viruses such as adenovirus and AAV are suitable for incorporation into the disclosed particles as well. The present example details preparation of one exemplary lentivirus.

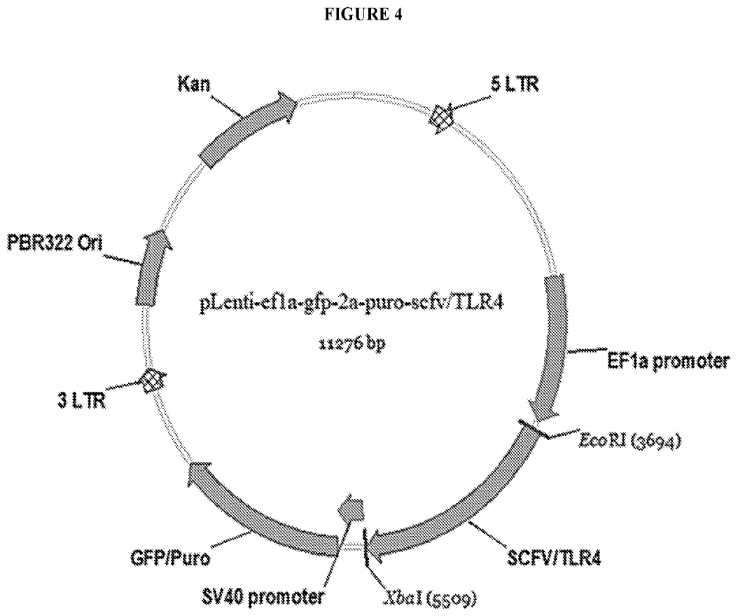

A lentiviral vector (shown in FIG. 4) was packaged into lentiviral particles using the third generation packaging mix (Applied Biological Materials Inc., Richmond, Canada) in 293 FT cells according to the manufacturer's protocol. Lentiviral particles were purified from supernatant using a PuRetro Lentivirus Purification kit and the titers were determined with a qPCR lentivirus titration kit (Applied Biological Materials Inc., Richmond, Canada).

### Example 2—Establishment of an Expression Cell Line

Various cell lines may be used to express the non-infective viruses (e.g., lentivirus) that are attached to the base particle. The present example details the creation of an exemplary expression cell line.

Human monocytic THP-1 cells were cultured in 12 well plates in RPMI Medium 1640 medium supplemented with 10% FBS and antibiotics. THP-1 cells were transduced with lentiviral particles at a MOI of 10 in the presence of polybrene (8 μg/ml). Viral infected THP-1 cells were selected with puromycin (1 ug/nil) to establish a THP-1 expression cell line.

### Example 3—CTLA Stimulation by the Disclosed Particles

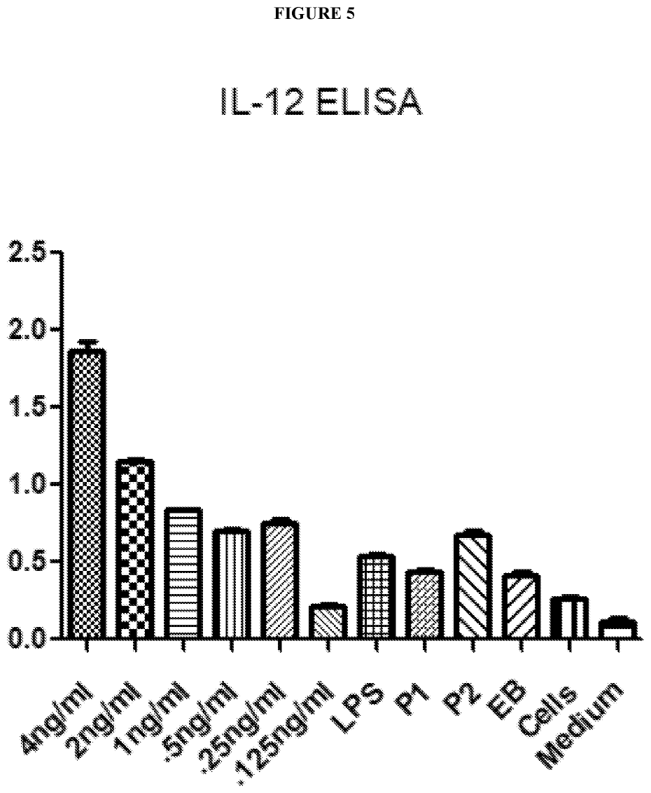

THP-1 cells expressing a chimeric receptor comprising an anti-CTLA4 scFv, a CD8 transmembrane domain, and a TLR4 intracellular domain (the nucleic acid sequence is shown in FIG. 1 and the amino acid sequence is shown in FIG. 2) were cultured in 24 well plates and recombinant CTLA4 protein was added into the culture at 500 ng/ml. After overnight culture, supernatants were collected for the measurement of pro-inflammatory cytokines, specifically IL-12, was performed by ELISA. Lipopolysaccharide (LPS) is a classic stimulator of TLRs and was used as the positive control at a concentration of 10 ng/ml. The results for this experiment are shown in FIG. 5.

As indicated in FIG. 5, addition of recombinant CTLA4 resulted in a concentration-dependent increase in expression of IL-12, indicating a strong immune activation response in the cells expressing the chimeric receptor. These results suggest that binding of the chimeric receptor to its target (CTLA4) would likewise stimulate production of other M1-type cytokines, such as IFN-α, IRN-γ, TNF-α, IL-6, and/or IL-1β.

### Example 4—Prophetic In Vivo Study

C57 B6 mice are injected with 1×10⁶ B16 murine melanoma cells. After twelve (12) days, the mice have palpable xenograft tumors.

Twelve days after the injection of the B16 murine melanoma cells, mice are treated with one of two delivery vectors. Control mice receive an intradermal injection of 1×10⁶ delivery vectors containing 1×10⁷ green fluorescence protein (GFP)-expressing adenovirus as the virus component of the vector. Mice in the experimental group receive an intradermal injection of 1×10⁶ delivery vectors containing 1×10⁷ adenovirus designed to express a chimeric receptor comprising an anti-CTLA4 scFv, a linker, a CD8 alpha chain hinge and transmembrane domain, and a cytoplasmic TLR4 domain.

The volume of each mouse's tumor is measured following the single dose treatment. All mice in the control group are expected to die on or before day 28 post treatment. All mice in the experimental group that receive the chimeric receptor-expressing delivery vector are expected to survive beyond day 45 post treatment, and their tumor volumes are expected to decrease.

One skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

1. A delivery vector comprising: (i) a base particle and (ii) a non-infectious virus attached to the outside of the particle, wherein the non-infectious virus comprises a nucleic acid encoding a chimeric receptor comprising a target binding domain, a transmembrane domain, and an intracellular signaling domain.

2. The delivery vector of claim 1, wherein the target binding domain of the chimeric receptor comprises an scFv that binds to an immune checkpoint protein.

3. The delivery vector of claim 2, wherein the checkpoint protein is selected from the group consisting of CTLA-4, PD-1, PD-L1, LAG3, B7.1, B7-H3, B7-H4, TIM3, VISTA, CD137, OX40, CD40, CD27, CCR4, GITR, NKG2D, and KIR.

4. The delivery vector of claim 3, wherein the checkpoint protein is CTLA-4.

5. The delivery vector of claim 4, wherein the target binding domain comprises an scFv comprising SEQ ID NO: 3 or SEQ ID NO: 3 with the IgK leader sequence removed.

6. The delivery vector of claim 4, wherein the target binding domain comprises a variable heavy chain sequence of SEQ ID NO: 1 and a variable light chain sequence of SEQ ID NO: 2.

7. The delivery vector of claim 3, wherein the checkpoint protein is PD-1.

8. The delivery vector of claim 7, wherein the target binding domain comprises a variable heavy chain sequence and a variable light chain sequence corresponding to the respective variable heavy and light chain sequences of pembrolizumab, nivolumab, cemiplimab, spartalizumab, camrelizumab, or sintilimab.

9. The delivery vector of claim 3, wherein the checkpoint protein is PD-L1.

10. The delivery vector of claim 9, wherein the target binding domain comprises a variable heavy chain sequence and a variable light chain sequence corresponding to the respective variable heavy and light chain sequences of durvalumab, atezolizumab or avelumab.

11. The delivery vector of claim 1, wherein the target binding domain is specific for OX40.

12. The delivery vector of claim 11, wherein the target binding domain comprises an scFv comprising a variable heavy chain sequence and a variable light chain sequence corresponding to the respective variable heavy and variable light chain sequences of scFv may comprise the CDRs and/or variable domain regions of 9B12 (NCT01644968), MOXR0916, PF-04518600, MEDI0562, MEDI6469, MEDI6383, PF-04518600, or BMS 986178.

13. The delivery vector of claim 11, wherein the target binding domain comprises an extracellular domain of OX40L.

14. The delivery vector of claim 1, wherein the transmembrane domain comprises at least the transmembrane portion of a toll-like receptor, CD28, CD4, CD8, 4-1BB, CD27, ICOS, OX40, HVEM, or CD30.

15. The delivery vector of claim 1, wherein the transmembrane domain comprises any one of SEQ ID NOs: 16-25.

16. The delivery vector of claim 1, wherein the intracellular signaling domain comprises an intracellular domain of a toll-like receptor (TLR).

17. The delivery vector of claim 16, wherein the TLR is TLR4 or TLR 9.

18. The delivery vector of claim 1, wherein the intracellular signaling domain comprises SEQ ID NO: 26 or SEQ ID NO: 27.

19. The delivery vector of claim 1, wherein the non-infectious virus is an adenovirus.

20. The delivery vector of claim 19, wherein the adenovirus is a recombinant adenovirus.

21. The delivery vector of claim 1, wherein the non-infectious virus is also non-replicative.

22. The delivery vector of claim 1, wherein the nucleic acid encoding the chimeric receptor is comprised within an expression vector.

23. The delivery vector of claim 22, wherein the expression vector comprises a T7 promoter.

24. The delivery vector of claim 22, wherein the expression vector comprises a hypoxia-induced promoter.

25. The delivery vector of claim 22, wherein the expression vector comprises SEQ ID NO: 44.

26. The delivery vector of claim 1, wherein the base particle is a yeast cell wall particle (YCWP).

27. The delivery vector of claim 26, wherein the YCWP is loaded with a biological material.

28. The delivery vector of claim 27, wherein the biological material is a tumor lysate.

29. The delivery vector of claim 1, wherein the base particle is a bead.

30. The delivery vector of claim 29, wherein the bead is a ferro-magnetic particle, a microbead, or a microsphere.

31. The delivery vector of claim 1, wherein the delivery vector is a size that allows it to be preferentially phagocytized by a monocytic cell.

32. The delivery vector of claim 31, wherein the monocytic cell is a macrophage.

33. The delivery vector of claim 32, wherein the macrophage is a tumor-associated macrophage (TAM).

34. A method of treating cancer in a patient comprising administering to a patient with cancer the delivery vector of claim 1.

35. The method of claim 34, wherein the delivery vector is administered intradermally.

36. The method of claim 34, wherein the delivery vector is administered proximate to a target lymph node.

37. The method of claim 34, wherein the cancer comprises at least one tumor comprising a hypoxic microenvironment.

38. The method of claim 34, wherein the at least one tumor comprises tumor-associated macrophages (TAMs).

39. The method of claim 34, wherein the delivery vector is phagocytosed by a macrophage and the macrophage subsequently expresses the chimeric receptor on its surface.

40. A method of stimulating the immune system in a patient comprising administering to a patient with cancer the delivery vector of claim 1.

41. The method of claim 40, wherein the delivery vector is administered intradermally.

42. The method of claim 40, wherein the delivery vector is administered proximate to a target lymph node.

43. A monocytic cell comprising a chimeric receptor expressed on its surface, the chimeric receptor comprising a target binding domain, a transmembrane domain, and an intracellular domain.

44. The monocytic cell of claim 43, wherein the cell is a macrophage or a dendritic cell.

45. The monocytic cell of claim 43, wherein the target binding domain of the chimeric receptor comprises an scFv that binds to an immune checkpoint protein.

46. The monocytic cell of claim 45, wherein the checkpoint protein is selected from the group consisting of CTLA-4, PD-1, PD-L1, LAG3, B7.1, B7-H3, B7-H4, TIM3, VISTA, CD137, OX40, CD40, CD27, CCR4, GITR, NKG2D, and KIR.

47. The monocytic cell of claim 45, wherein the checkpoint protein is CTLA-4, PD-1, or PD-L1.

48. The monocytic cell of claim 43, wherein the target binding domain comprises an scFv comprising SEQ ID NO: 3 or SEQ ID NO: 3 with the IgK leader sequence removed.

49. The monocytic cell of claim 43, wherein the target binding domain comprises a variable heavy chain sequence and a variable light chain sequence corresponding to the respective variable heavy and light chain sequences of ipilimumab, tremelimumab, pembrolizumab, nivolumab, cemiplimab, spartalizumab, camrelizumab, sintilimab, durvalumab, atezolizumab or avelumab.

50. The monocytic cell of claim 43, wherein the target binding domain of the chimeric receptor is specific for OX40.

51. The monocytic cell of claim 50, wherein the target binding domain comprises an scFv comprising a variable heavy chain sequence and a variable light chain sequence corresponding to the respective variable heavy and variable light chain sequences of scFv may comprise the CDRs and/or variable domain regions of 9B12 (NCT01644968), MOXR0916, PF-04518600, MEDI0562, MEDI6469, MEDI6383, PF-04518600, or BMS 986178.

52. The monocytic cell of claim 50, wherein the target binding domain comprises an extracellular domain of OX40L.

53. The monocytic cell of claim 43, wherein the transmembrane domain comprises at least the transmembrane portion of a toll-like receptor, CD28, CD4, CD8, 4-1BB, CD27, ICOS, OX40, HVEM, or CD30.

54. The monocytic cell of claim 43, wherein the transmembrane domain comprises any one of SEQ ID NOs: 16-25.

55. The monocytic cell of claim 43, wherein the intracellular signaling domain comprises an intracellular domain of a toll-like receptor (TLR).

56. The monocytic cell of claim 55, wherein the TLR is TLR4 or TLR 9.

57. The monocytic cell of claim 43, wherein the intracellular signaling domain comprises SEQ ID NO: 26 or 27.

58-63. (canceled)
